(19) United States
(12) Patent Application Publication
(10) Pub. No.: US 2026/0137304 A1
(43) Pub. Date: May 21, 2026

(54) ELECTRONIC DEVICE AND WEARABLE DEVICE FOR PROVIDING WALKING ABILITY EVALUATION MODE, AND METHODS FOR OPERATING THEM

(71) Applicants: SAMSUNG ELECTRONICS CO., LTD., Suwon-si (KR); SAHMYOOK UNIVERSITY INDUSTRY-ACADEMIC COOPERATION FOUNDATION, Seoul (KR)

(72) Inventors: Hwangjae LEE, Suwon-si (KR); Dokwan LEE, Suwon-si (KR); Wan Hee LEE, Seoul (KR); Dongwoo KIM, Suwon-si (KR); Jang Hoon SHIN, Seoul (KR)

(21) Appl. No.: 19/447,846

(22) Filed: Jan. 13, 2026

Related U.S. Application Data
(63) Continuation of application No. PCT/KR2025/009032, filed on Jun. 27, 2025.

(30) Foreign Application Priority Data
Jul. 26, 2024 (KR) 10-2024-0099752
Sep. 13, 2024 (KR) 10-2024-0125322

(51) Int. Cl. A61B 5/11; A61B 5/00

(52) U.S. Cl. CPC A61B 5/112; A61B 5/7405; A61B 5/742; A61B 5/6803; A61B 5/681; A61B 2562/0219

(57) ABSTRACT

A wearable device and electronic device that provide a walking ability evaluation mode and/or an operating method thereof are provided. The operating method of the electronic device may include receiving a user input for a walking ability evaluation, transmitting a control signal to activate the walking ability evaluation mode to a wearable device in response to the receiving of the user input, receiving sensor data comprising motion information on a motion of the wearable device corresponding to a body motion of the user from the wearable device in response to the transmitting of the control signal, determining evaluation data on walking posture of the user based on the received sensor data, and performing at least one operation of determining a recommended exercise program and outputting guide content based on the determined evaluation data on the walking posture.

### CROSS-REFERENCE TO RELATED APPLICATIONS

This application is a continuation application of International Application No. PCT/KR2025/009032 designating the United States, filed on Jun. 27, 2025, in the Korean Intellectual Property Receiving Office and claiming priority to Korean Patent Application No. 10-2024-0099752, filed on Jul. 26, 2024, and Korean Patent Application No. 10-2024-0125322, filed on Sep. 13, 2024, the disclosures of which are all hereby incorporated by reference herein in their entireties.

### BACKGROUND

### Technical Field

Certain example embodiments may relate to a wearable device and/or an electronic device providing a walking ability evaluation mode and/or an operating method thereof.

### Background Art

A walking assistance device generally refers to a machine or a device that helps a patient (e.g., a patient unable to walk on their own because of diseases, accidents, or other causes) with walking exercises for rehabilitation treatment and/or exercise, and/or helps a person with their workouts. In our current, rapidly aging society, a growing number of people experience inconvenience when walking or have difficulty with normal walking due to malfunctioning joints, and there is increasing interest in walking assistance devices. A walking assistance device is worn on a user's body to assist the user by providing the necessary muscular strength and/or to assist the user with their exercises and/or walking by inducing the user to walk such that the user may walk in a normal walking pattern. The walking assistance device may also assist the user with various leg exercises (e.g., power walking, jogging, stair climbing, lunging, and stretching).

The above information may be presented as the related art to help with the understanding of the disclosure. Any of the above description is not claimed as the prior art related to the present disclosure or is not used to determine the prior art.

### SUMMARY

According to certain example embodiments, an electronic device may include a communication circuit configured to receive sensor data that is measured by one or more sensors of a wearable device from the wearable device worn on a body of the user, a memory configured to store instructions, and one or more processors. The instructions, when executed by the one or more processors comprising processing circuitry, may cause the one or more processors to individually and/or collectively determine evaluation data on a walking posture of the user based on the received sensor data and perform at least one operation determining a recommended exercise program and outputting guide content based on the determined evaluation data on the walking posture of the user. The sensor data may include motion information on motion of the wearable device corresponding to a body motion of the user. The evaluation data the walking posture of the user may include evaluation data on a pelvis motion of a pelvis of the user. The evaluation data on the pelvis motion may include one or a combination of evaluation data on rotation of the pelvis, evaluation data on an anterior-posterior tilt of the pelvis, or evaluation data on a lateral oblique of the pelvis.

According to certain example embodiments, a wearable device may include one or more sensors configured to acquire sensor data comprising motion information on the wearable device by measuring a motion of the wearable device corresponding to a motion of a user wearing the wearable device 100, a communication circuit configured to transmit the sensor data, a memory configured to store instructions, and one or more processors comprising processing circuitry. The instructions, when executed by the one or more processors individually and/or collectively, may cause the one or more processors to, in a walking ability evaluation mode, control the communication circuit to transmit the sensor data to an electronic device and enable the electronic device to determine evaluation data on the walking posture of the user based on the sensor data and perform at least one operation of determining a recommended exercise program and outputting guide content based on the determined evaluation data on the walking posture.

According to an example embodiment, an operating method of an electronic device may include receiving a user input for a walking ability evaluation, transmitting a control signal to activate the walking ability evaluation mode to a wearable device in response to the receiving of the user input, receiving sensor data including motion information on a motion of the wearable device corresponding to a body motion of the user from the wearable device in response to the transmitting of the control signal, determining evaluation data on a walking posture of the user based on the received sensor data, and performing at least one operation of determining a recommended exercise program and outputting guide content based on the determined evaluation data on the walking posture.

### BRIEF DESCRIPTION OF DRAWINGS

These and/or other aspects, features and advantages will become apparent and more readily understood from the following description of exemplary embodiments taken in conjunction with the accompanying drawings.

FIG. 1 is a diagram illustrating an overview of a wearable device worn on a user's body, according to various example embodiments.

FIG. 2 is a diagram illustrating an exercise assistance system according to various example embodiments.

FIG. 3 is a rear schematic view illustrating a wearable device according to various example embodiments.

FIG. 4 is a left side view illustrating a wearable device worn on a user's body, according to various example embodiments.

FIG. 5 is a diagram illustrating components of an electronic system of a wearable device, according to various example embodiments.

FIG. 6 is a diagram illustrating an interaction between a wearable device and an electronic device, according to various example embodiments.

FIG. 7 is a diagram illustrating components of an electronic device, according to various example embodiments.

FIG. 8 is a diagram illustrating an operating method of a wearable device and electronic device for assessing the user's walking ability, according to various example embodiments.

FIG. 9 is a diagram illustrating user interface screens provided to a user through an electronic device when walking ability evaluation is performed, according to various example embodiments.

FIG. 10 is a diagram illustrating assessing the user's walking ability by using a wearable device and electronic device, according to various example embodiments.

FIG. 11 is a diagram illustrating an example of walking analysis result content according to various example embodiments.

FIG. 12 is a flowchart illustrating operations of a walking ability evaluation process by using a wearable device and electronic device, according to various example embodiments.

FIG. 13 is a diagram illustrating measuring a user's stride and gait speed based on sensor data, according to various example embodiments.

FIG. 14 is a diagram illustrating measuring a user's gait cycle based on sensor data, according to various example embodiments.

FIGS. 15A, 15B, and 15C are diagrams each illustrating determining evaluation data on a user's pelvis motion based on sensor data, according to various example embodiments.

FIG. 16 is a flowchart illustrating operations of a method of providing a recommended exercise program based on a walking ability evaluation result, according to various example embodiments.

FIG. 17 is a diagram illustrating a recommended exercise program provided based on a walking ability evaluation result, according to various example embodiments.

FIG. 18 is a diagram illustrating voice coaching content provided based on a walking ability evaluation result, according to various example embodiments.

### DETAILED DESCRIPTION

Hereinafter, embodiments will be described in detail with reference to the accompanying drawings. When describing the examples with reference to the accompanying drawings, like reference numerals refer to like elements and a repeated description related thereto will be omitted.

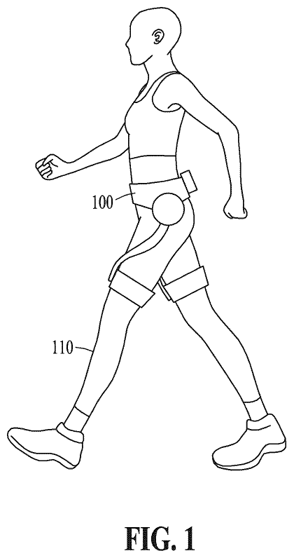

FIG. 1 is a diagram illustrating an overview of a wearable device worn on a user's body, according to various embodiments.

Referring to FIG. 1, in an embodiment, a wearable device 100 may be a device worn on the body of a user 110 to assist the user 110 in walking, exercising, and/or working. The wearable device 100 may be used to measure physical ability (e.g., walking ability, exercise ability, or an exercise posture) of the user 110. In specific embodiments, the term “wearable device” may be replaced with “wearable robot,” “walking assistance device,” or “exercise assistance device”. The user 110 may be a person who walks, exercises, or performs a task while wearing the wearable device 100.

In some embodiments, the description is provided based on an example of the hip-type wearable device 100 as shown in FIG. 1. However, the range of embodiments are not limited thereto. As described above, the wearable device 100 may also be worn on other body parts (e.g., the upper arms, lower arms, hands, calves, or feet) other than the waist and thighs. The shape and configuration of the wearable device 100 may vary depending on the body part on which the wearable device 100 is worn.

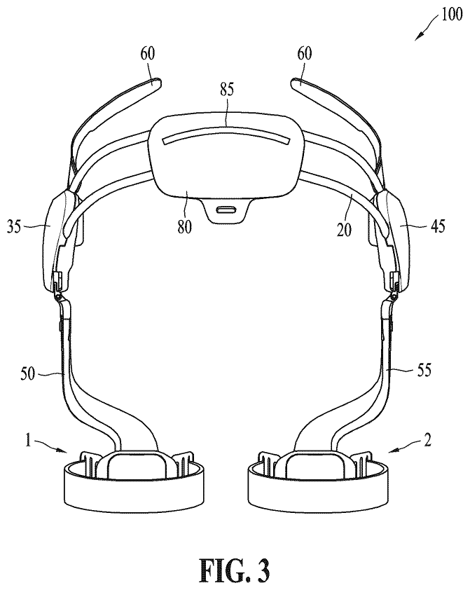

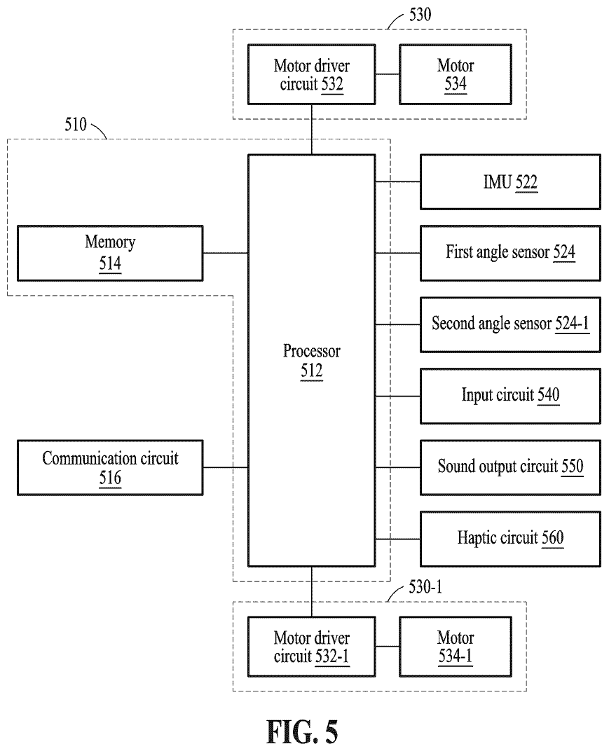

The wearable device 100 may include a support frame (e.g., a waist support frame 20 of FIG. 3) configured to support the body of the user 110 when the wearable device 100 is worn on the body of the user 110, a driving module (e.g., a first driving module 45 and a second driving module 35 of FIG. 3) configured to generate torque to be applied to the legs of the user 110, a torque transmission frame (e.g., a first torque transmission frame 55 and a second torque transmission frame 50 of FIG. 3) configured to transmit the torque generated by the driving module to the legs of the user 110, a sensor circuit including one or more sensors configured to acquire sensor data including motion information on a body motion (e.g., a leg motion and a pelvic motion) of the user 110, a control circuit (e.g., a control circuit 510 of FIG. 5) configured to control an operation of the wearable device 100, and a battery configured to supply power to each component of the wearable device 100.

In an embodiment, the sensor circuit of the wearable device 100 may include an angle sensor (e.g., a first angle sensor 524 and a second angle sensor 524-1 of FIG. 5) and an inertial measurement unit (IMU) (e.g., an IMU 522 of FIG. 5). The angle sensor may measure a rotation angle of the torque transmission frame of the wearable device 100 corresponding to a hip joint angle of the user 110. The angle sensor may include, for example, an encoder and/or a hall sensor. In an embodiment, the angle sensor may be disposed adjacent to a position where a motor included in the driving module is connected directly or indirectly to the torque transmission frame. The IMU may include an acceleration sensor, a gyroscope, and a magnetometer and may measure a change in acceleration and/or angular velocity according to the motion of the user 110. The inertia sensor may measure the motion of the waist support frame or a base body (e.g., a base body 80 of FIG. 3) of the wearable device 100, for example. The motion of the waist support frame or the base body measured by the IMU may correspond to a pelvic motion (or an upper body motion) of the user 110.

In an embodiment, the IMU, the control circuit, a peripheral circuit (e.g., a sound output circuit, a communication circuit, or a haptic circuit), and a battery may be disposed in the base body of the wearable device 100. The base body may be on the waist of the user 110 when the user 110 wears the wearable device 100. The base body may be formed on or attached to the outside of the waist support frame of the wearable device 100. The base body may support the lumbar region of the user 110.

The wearable device 100 may be worn on a body (e.g., a lower body (the legs, ankles, knees, etc.) and/or an upper body (the torso, arms, wrists, etc.)) of the user 110 to apply an external force such as an assistance force and/or a resistance force to a body motion of the user 110. The assistance force may be a force assisting the body motion of the user 110, which is applied in the same direction as a direction of the body motion of the user 110. The resistance force may be a force applied in a direction opposite to the body motion direction of the user 110, the force hindering a body motion of the user 110. The term “resistance force” may also be referred to as “exercise load”.

In an embodiment, the wearable device 100 may operate in a walking assistance mode for assisting the walking of the user 110. In the walking assistance mode, the wearable device 100 may assist the walking of the user 110 by applying an assistance force generated through a driving module (including a motor) of the wearable device 100 to the body of the user 110. The wearable device 100 may expand the walking ability of the user 110 by allowing the user 110 to walk independently or walk for a long time by providing a force needed for the walking of the user 110. The wearable device 100 may help improve the walking of a user having an abnormal walking habit or gait posture.

In an embodiment, the wearable device 100 may operate in an exercise assistance mode for enhancing the effect of exercise of the user 110 or providing various exercise experiences to the user 110. The exercise assistance mode may include a resistance mode and an assistance mode. The resistance mode of the exercise assistance mode may be a mode for hindering a body motion of the user 110 or providing resistance to a body motion of the user 110 by applying a resistance force generated by the driving module to the body of the user 110. When the wearable device 100 is a hip-type wearable device that is worn on the waist (or pelvis) and legs (e.g., thighs) of the user 110, the wearable device 100 may provide an exercise load to a leg motion of the user 110 while being worn on the legs in the resistance mode, thereby enhancing the effect of exercise on the legs of the user 110. The assistance mode of the exercise assistance mode may be a mode for applying an assistance force to assist with a body motion of the user 110 to the body of the user 110. In the assistance mode, an assistance force in the same direction as a body motion may be provided to the user 110. For example, when a person with a disability or an elderly person wears the wearable device 100 to exercise, the wearable device 100 may provide an assistance force to assist a body motion. In the assistance mode, the wearable device 100 may provide a force in the same direction as a direction of a leg motion of the user 110, and the user 110 may exercise with a small force through the force provided by the wearable device 100. In an exercise program performed using the wearable device 100, the resistance mode and the assistance mode may be combined and operated. For example, the wearable device 100 may provide an assistance force and a resistance force in combination for each exercise session or time interval in such a manner of providing an assistance force in one exercise session and providing a resistance force in another exercise session.

In the exercise assistance mode, various exercise programs may be operated depending on the exercise purpose or physical ability of the user 110. The exercise program may be exercise content that the user 110 performs using the wearable device 100 and may include, for example, cardio exercises, strength training, posture balancing, or any combination thereof. The types of exercise programs are not limited thereto and may vary. The resistance mode and the assistance mode may be alternately activated appropriately depending on an exercise program performed by the wearable device 100, and a target exercise speed that is suitable to a physical condition (e.g., a heart rate) of the user 110 may be guided to the user during the exercise of the user 110.

In an embodiment, the wearable device 100 may operate in a physical ability measurement mode to measure the physical ability of the user 110. The wearable device 100 may measure motion information of the user 110 using sensors (e.g., an angle sensor and an inertial measurement unit (IMU)) provided in the wearable device 100 during walking and/or exercise of the user 110 and may evaluate the physical ability of the user 110 based on the measured motion information. A gait index (e.g., the number of steps, the total walking distance, or a stride length) and/or an exercise ability indicator (e.g., muscular strength, exercise endurance, or posture balance) of the user 110 may be estimated through the motion information of the user 110 measured by the wearable device 100.

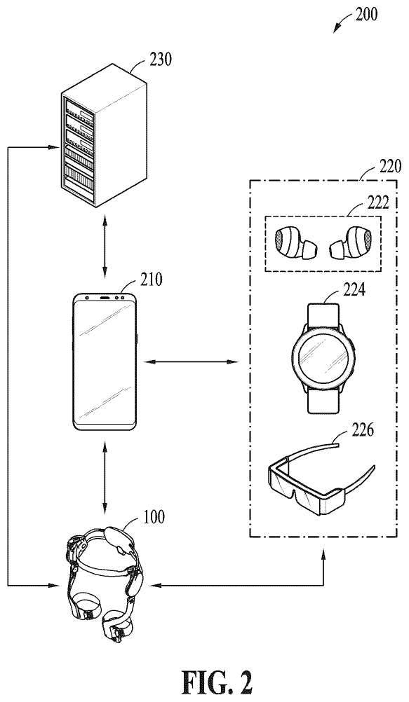

In an embodiment, a physical ability measurement mode may include a walking ability evaluation mode (or a walking ability measurement mode) to evaluate (or measure) the walking ability of the user 110. In the walking ability evaluation mode, a user's walking posture and/or walking activeness may be evaluated. The evaluation of the walking posture may include, for example, the evaluation of a pelvic motion during walking and/or the evaluation of a gait symmetry between left and right steps of the user 110. The evaluation of the walking activeness may include, for example, the evaluation of a gait speed, a stride, and/or a gait cycle of the user 110. The walking ability evaluation results may be provided to a user through the wearable device 100 and/or another electronic device (e.g., the electronic device 210 of FIG. 2 or another wearable device 220). For example, the walking ability evaluation results may be converted into voice data through a text-to-speech (TTS) function and may be output through a speaker of the wearable device 100 or a speaker of another wearable device (e.g., wireless earphones or a smartwatch) worn by the user 110. The walking ability evaluation results may also be provided visually to the user 110 via a display of the other electronic device. The walking ability evaluation mode is described in more detail below.

FIG. 2 is a diagram illustrating an exercise assistance system according to various embodiments.

Referring to FIG. 2, an exercise assistance system 200 may include the wearable device 100, an electronic device 210, another wearable device 220, and a server 230. At least one (e.g., the electronic device 210, the other wearable device 220, or the server 230) of the devices excluding the wearable device 100 may be omitted from the exercise assistance system 200, or at least one device (e.g., a dedicated controller for the wearable device 100) may be added to the exercise assistance system 200.

In an embodiment, the wearable device 100 may be worn on the body of a user to assist a motion of the user in a walking assistance mode. For example, the wearable device 100 may be worn on the legs of the user to help the user in walking by generating an assistance force for assisting a leg motion of the user.

In an embodiment, the wearable device 100 may generate a resistance force for hindering a body motion of the user and/or an assistance force for assisting a body motion of the user and apply the generated resistance force and/or assistance force to the body of the user to enhance the effect of exercise of the user in the exercise assistance mode. In the exercise assistance mode, the user may select an exercise program (e.g., cardio exercise such as power walking and outdoor walking, strength training such as squats, split lunges, dumbbell squats, and lunge and knee ups, a stretching exercise, a posture balancing exercise, or any combination thereof) that the user desires to conduct using the wearable device 100 through the electronic device 210 and/or an exercise intensity applied to the exercise program. The wearable device 100 may control a driving module of the wearable device 100 based on the exercise program and/or the exercise intensity selected by the user. The wearable device 100 may adjust the strength of the resistance force and/or the assistance force generated by the driving module based on the exercise intensity selected by the user. The wearable device 100 may control the driving module to generate a resistance force corresponding to the exercise intensity selected by the user. As the exercise intensity increases, the strength of the resistance force applied to the user may increase.

In an embodiment, the wearable device 100 may be used to measure the physical ability (e.g., the walking ability) of the user by interoperating with the electronic device 210. The wearable device 100 may operate in a physical ability measurement mode, that is, a mode for measuring the physical ability of the user, by control of the electronic device 210 and may transmit sensor data including the motion information of the wearable device 100 according to the physical motion of the user in the physical ability measurement mode to electronic device 210. The electronic device 210 may evaluate the physical ability of the user by analyzing the sensor data received from the wearable device 100 and may provide the evaluation results to the user. The electronic device 210 may recommend an exercise program that may be beneficial to the user based on the evaluation results of physical ability or may provide a guide voice during an exercise. For example, if the gait speed is evaluated poorly in the evaluation of the walking ability, the electronic device 210 may recommend an exercise program (e.g., a power walking program) to improve the user's gait speed or may output a guide voice (e.g., “Shall we walk a little faster?”) to induce improvement in the user's gait speed while the user is walking.

The wearable device 100 may transmit sensor data measured through an angle sensor and/or an IMU and the device information (e.g., charging state information, operation mode information, setting information) of the wearable device 100 to the electronic device 210 and/or the server 230 and may receive a control signal for controlling the operation of the wearable device 100 from the electronic device 210 and/or the server 230.

The electronic device 210 may communicate with the wearable device 100 via wireless communication (e.g., Bluetooth communication) or wired communication, may remotely control the wearable device 100, or may provide state information on a state (e.g., a booting state, a charging state, an exercise program operation state, or an error state) of the wearable device 100 to the user. The electronic device 210 may recommend an exercise program conducted by using the wearable device 100 to the user and may analyze an exercise performed by the user. The electronic device 210 may receive, from the wearable device 100, sensor data acquired by a sensor (e.g., the angle sensor or the IMU) of the wearable device 100 and may estimate a current exercise state, an exercise result, an exercise posture, and/or physical ability of the user based on the received sensor data. The electronic device 210 may provide the user with the estimated current exercise state, exercise result, exercise posture, and/or physical ability of the user through a graphical user interface (GUI).

In an embodiment, the user may execute a program (e.g., an application) on the electronic device 210 to control the wearable device 100, and adjust an operation or a setting value (e.g., the magnitude of torque output from a motor of a driving module, the volume of audio output from a sound output circuit (e.g., a sound output circuit 550 of FIG. 5), or the brightness of a lighting module (e.g., a lighting module 85 of FIG. 3) of the wearable device 100 through the corresponding program. The program executed by the electronic device 210 may provide the GUI for an interaction with the user. The electronic device 210 may be a device in various forms. For example, the electronic device 210 may include a portable communication device (e.g., a smartphone), a computer device, a portable multimedia device, or a home appliance (e.g., a television, an audio device, or a projector device), but is not limited thereto.

According to an embodiment, the electronic device 210 may be connected to the server 230 using short-range wireless communication or cellular communication. The server 230 may receive user profile information of the user who uses the wearable device 100 from the electronic device 210, and store and manage the received user profile information. The user profile information may include, for example, information on at least one of the name, age, gender, height, weight, medical history, or body mass index (BMI). The server 230 may receive exercise history information on an exercise performed by the user from the electronic device 210, and store and manage the received exercise history information. The server 230 may provide the electronic device 210 with various exercise programs or physical ability measurement programs to be provided to the user. In an embodiment, the server 230 may be connected to the wearable device 100. The server 230 may receive, from the wearable device 100, the sensor data measured by the wearable device 100 and transmit, to the wearable device 100, a control signal for controlling an operation of the wearable device 100 and/or data related to the exercise program. In an embodiment, the server 230 may be a cloud server.

According to an embodiment, the wearable device 100 and/or the electronic device 210 may be connected directly or indirectly to the other wearable devices 220. Exercise result information, physical ability information, and/or exercise posture evaluation information of the user that are determined by the electronic device 210 may be transmitted to the other wearable device 220 and provided to the user through the other wearable device 220. State information of the wearable device 100 may also be transmitted to the other wearable device 220 and provided to the user through the other wearable device 220. In an embodiment, the wearable device 100, the electronic device 210, and the other wearable device 220 may be connected to each other through wireless communication (e.g., Bluetooth communication or Wi-Fi communication). The other wearable device 220 may be, for example, wireless earphones 222, a smartwatch (or a watch-type wearable device) 224, or smart glasses (a wearable device in the type of glasses or goggles) 226 but is not limited thereto.

In an embodiment, the wireless earphones 222 may be wirelessly connected to the electronic device 210 and/or the wearable device 100 and may output a guiding voice, music, and/or sound effect related to the exercise program. The wireless earphones 222 may provide a guide voice to provide information (e.g., introduction of the exercise program or a remaining exercise time) related to the exercise program and/or a guide voice for real-time exercise coaching to the user. The wireless earphones 222 may include a microphone, and the microphone may receive a voice input from the user. The voice input received through the microphone may be transmitted to the electronic device 210, and voice recognition for the voice input may be performed by the electronic device 210.

In an embodiment, the smartwatch 224 may include a biometric sensor (e.g., a heart rate sensor or an electromyography sensor) configured to measure a biosignal, including heart rate information of the user, and transmit the biosignal measured by the biometric sensor to the electronic device 210 and/or the wearable device 100. For example, the electronic device 210 may estimate the heart rate information (e.g., a current heart rate, a maximum heart rate, and an average heart rate) and/or electromyography information of the user based on the biosignal received from the smartwatch 224 and may provide the user with the estimated heart rate information and/or electromyography information.

In an embodiment, the smartwatch 224 may include an IMU configured to measure motion information of the user and/or a position sensor configured to measure position information of the user and may transmit the motion information and/or position information of the user to the electronic device 210 and/or the wearable device 100. The smartwatch 224 may include a communication circuit (e.g., a short-range communication circuit) for communicating with another device (e.g., the electronic device 210 or the wearable device 100). In an embodiment, the smartwatch 224 may provide an interface related to an exercise program through a display. The interface related to the exercise program may be implemented through a separate application installed on the smartwatch 224. The user may also control the wearable device 100 through the smartwatch 224.

In an embodiment, the smart glasses 226 may provide information to the user through a display in the form of glasses. For example, the smart glasses 226 may output information on the current exercise speed, a target exercise speed, the currently achieved exercise amount, the exercise time, and/or the biometric information, through the display in an exercise mode. In addition, the smart glasses 226 may output a screen for guiding an exercise path to the user.

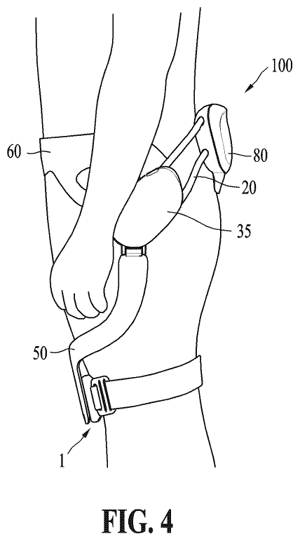

FIG. 3 is a rear schematic view illustrating a wearable device according to various embodiments. FIG. 4 is a left side view illustrating a wearable device worn on a user's body, according to various embodiments.

Referring to FIGS. 3 and 4, the wearable device 100 according to an embodiment may include the base body 80, the waist support frame 20, the driving modules 35 and 45, the torque transmission frames 50 and 55, thigh fastening portions 1 and 2, and a waist fastening portion 60. In an embodiment, at least one of the components described above may be omitted from the wearable device 100, or one or more other components may be added to the wearable device 1000.

The base body 80 may be on the waist of a user when the user wears the wearable device 100. The base body 80 worn on the waist of the user may cushion and support the waist of the user. The base body 80 may be above the hip of the user when the user wears the wearable device 100 such that the wearable device 100 may not deviate downward due to gravity or reduce the possibility of deviation. The base body 80 may distribute some of the weight of the wearable device 100 to the waist of the user while wearing the wearable device 100. The base body 80 may be connected directly or indirectly to the waist support frame 20. Waist support frame connection elements (not shown) which may be connected directly or indirectly to the waist support frame 20 may be at both edges of the base body 80.

In an embodiment, at least one of a processor (e.g., a processor 512 of FIG. 5), a battery, a power management integrated circuit (PMIC) configured to convert power from the battery to an operating voltage of each component of the wearable device 100 and supply the converted power to each component, a memory (e.g., a memory 514 of FIG. 5), an IMU (e.g., the IMU 522 of FIG. 5), a communication circuit (e.g., a communication circuit 516 of FIG. 5), a sound output circuit (e.g., the sound output circuit 550 of FIG. 5), or a haptic circuit (e.g., a haptic circuit 560 of FIG. 5) may be positioned inside the base body 80. The base body 80 may protect the components inside the base body 80.

In an embodiment, a display (not shown) may be provided on the outer surface of the base body 80. The display may provide a screen for various pieces of visual information related to the wearable device 100 (e.g., state information of the wearable device 100) and a user interface (UI).

The waist support frame 20 may support a body part (e.g., the waist) of the user when the wearable device 100 is worn on the body of the user. The waist support frame 20 may extend from both edges of the base body 80. The waist of the user may be accommodated inside the waist support frame 20. The waist support frame 20 may include one or more rigid body beams. Each beam may be a curved shape with a preset curvature such that the beam may enclose the user's waist. The waist fastener 60 may be connected directly or indirectly to an edge of the waist support frame 20. The driving modules 35 and 45 may be directly or indirectly connected to the waist support frame 20.

In an embodiment, the wearable device 100 may include a sensor circuit including one or more sensors. The sensor circuit may include one or more sensors configured to acquire sensor data including motion information of the user and/or motion information of the components of the wearable device 100. For example, the one or more sensors may include the IMU (e.g., the IMU 522 of FIG. 5) configured to measure a pelvic motion of the user or the motion of the waist support frame 20 and/or an angle sensor (e.g., the first angle sensor 524 and the second angle sensor 524-1 of FIG. 5) configured to measure a hip joint angle of the user or an angle of a torque transmission frame (e.g., the first torque transmission frame 50 or the second torque transmission frame 55), but examples are not limited thereto. A hip joint angular velocity of the user or an angular velocity of the torque transmission frame may be determined by differentiating the hip joint angle of the user or the angle of the torque transmission frame measured by the angle sensor.

In an embodiment, the one or more sensors may further include at least one of a position sensor, a torque sensor, a pressure sensor, a temperature sensor, a biosignal sensor (e.g., a heart rate sensor or an electromyography sensor), a distance sensor, or a proximity sensor.

The waist fastening portion 60 may be connected, directly or indirectly, to the waist support frame 20 to fasten the waist support frame 20 to the waist of the user. The waist fastening portion 60 may include, for example, a pair of belts.

The first driving module 45 and the second driving module 35 may generate an external force (or torque) to be applied to the body of the user based on the control signal generated by the processor. For example, the first driving module 45 and the second driving module 35 may generate an assistance force or a resistance force applied to the legs of the user. In an embodiment, the first driving module 45 may be disposed at a position corresponding to the right hip joint of the user, and the second driving module 35 may be disposed at a position corresponding to the left hip joint of the user. The first driving module 45 may cause the first torque transmission frame 55 to move (or rotate) in the forward direction or backward direction of the wearable device 100 by generating a torque. The second driving module 35 may cause the second torque transmission frame 50 to move (or rotate) in the forward direction or backward direction of the wearable device 100 by generating a torque. The forward direction may be a direction corresponding to the front direction of the user or the flexion motion of a leg, and the backward direction may be a direction corresponding to the rear direction of the user or the extension motion of a leg.

The first driving module 45 may include a first actuator and a first joint member, and the second driving module 35 may include a second actuator and a second joint member. The first actuator may provide power to be transmitted to the first joint member, and the second actuator may provide power to be transmitted to the second joint member. The first actuator and the second actuator may each include a motor configured to generate power (or torque) by receiving power from the battery. When the motor is driven as the power is supplied thereto, the motor may generate a force (an assistance force) for assisting a body motion of the user or a force (a resistance force) for hindering a body motion of the user. In an embodiment, the processor may adjust the strength and direction of the force generated by the motor by adjusting a voltage and/or a current supplied to the motor.

In an embodiment, the first joint member and the second joint member may receive power from the first actuator and the second actuator, respectively, and apply an external force to the body of the user based on the received power. The first joint member and the second joint member may respectively be at positions corresponding to joints of the user. One side of the first joint member may be directly or indirectly connected to the first actuator, and the other side of the first joint member may be directly or indirectly connected to the first torque transmission frame 55. The first joint member may be rotated by the power received from the first actuator. An encoder or a Hall sensor that may operate as an angle sensor to measure a rotation angle (corresponding to a joint angle of the user) of the first joint member or the first torque transmission frame 55 may disposed on one side of the first joint member. One side of the second joint member may be connected to the second actuator, and the other side of the second joint member may be connected to the second torque transmission frame 50. The second joint member may be rotated by the power received from the second actuator. An encoder or a Hall sensor that may operate as an angle sensor to measure a rotation angle of the second joint member or the second torque transmission frame 50 may be disposed on one side of the second joint member.

In an embodiment, the first actuator may be disposed in a lateral direction of the first joint member, and the second actuator may be disposed in a lateral direction of the second joint member. A rotation axis of the first actuator and a rotation axis of the first joint member may be spaced apart from each other, and a rotation axis of the second actuator and a rotation axis of the second joint member may also be spaced apart from each other. However, the embodiments are not limited thereto, and an actuator and a joint member may share a rotation axis. In an embodiment, each actuator may be spaced apart from a corresponding joint member. In this case, each of the first driving module 45 and the second driving module 35 may further include a power transmission module (not shown) configured to transmit power from the actuator to the joint member. The power transmission module may be a rotary body, such as a gear, or a longitudinal member, such as a wire, a cable, a string, a spring, a belt, or a chain. However, the scope of the embodiment is not limited by the positional relationship between an actuator and a joint member and the power transmission structure described above.

In an embodiment, when the wearable device 100 is worn on the legs of the user, the first torque transmission frame 55 and the second torque transmission frame 50 may transmit torque generated by the first driving module 45 and the second driving module 35 to the body (e.g., the legs) of the user, respectively. The transmitted torque may function as an external force applied to a leg motion of the user. Respective ends of the first torque transmission frame 55 and the second torque transmission frame 50 may be directly or indirectly connected to the joint member and rotate. As the other ends of the first torque transmission frame 55 and the second torque transmission frame 50 are directly or indirectly connected to the first thigh fastening portion 2 and the second thigh fastening portion 1, the first torque transmission frame 55 and the second torque transmission frame 50 may transmit the torque generated by the first driving module 45 and the second driving module 35 to the thighs of the user while supporting the thighs of the user. For example, the first torque transmission frame 55 and the second torque transmission frame 50 may push or pull the thighs of the user. The first torque transmission frame 55 and the second torque transmission frame 50 may extend in a longitudinal direction of the thighs of the user or may be bent and enclose at least some portions of the circumferences of the thighs of the user. The first torque transmission frame 55 may be a torque transmission frame for transmitting torque to the right leg of the user, and the second torque transmission frame 50 may be a torque transmission frame for transmitting torque to the left leg of the user.

The first thigh fastening portion 2 and the second thigh fastening portion 1 may be directly or indirectly connected to the first torque transmission frame 55 and the second torque transmission frame 50, respectively, and fasten the wearable device 100 to the legs (specifically, thighs) of the user. The first thigh fastening portion 2 may be a thigh fastening portion to fix the first torque transmission frame 55 to a leg (e.g., a right thigh) of the user, and a second thigh fastening portion 1 may be a thigh fastening portion to fix the second torque transmission frame 50 to a leg (a left thigh) of the user.

In an embodiment, the first thigh fastener 2 may include a first cover, a first fastening frame, and a first strap, and the second thigh fastener 1 may include a second cover, a second fastening frame, and a second strap. The first cover and the second cover may apply torque generated by the first driving module 45 and the second driving module 35 to the thighs of the user, respectively. The first cover and the second cover may be disposed on respective sides of the thighs of the user and push or pull the thighs of the user, respectively. The first cover and the second cover may be disposed in the circumferential directions of the thighs of the user. The first cover and the second cover may extend to both sides from the other ends of the first torque transmission frame 55 and the second torque transmission frame 50 and may include curved surfaces corresponding to the thighs of the user. The respective ends of the first cover and the second cover may be directly or indirectly connected to the first fastening frame and the second fastening frame. The other ends of the first cover and the second cover may be directly or indirectly connected to the first strap and the second strap.

For example, the first fastening frame and the second fastening frame may be disposed to enclose at least some portions of the circumferences of the thighs of the user, thereby the thighs of the user may be prevented from, or chanced reduced of, being separated from the wearable device 100 or a possibility of separation may decrease. The first fastening frame may have a fastening structure that connects the first cover to the first strap, and the second fastening frame may have a fastening structure that connects the second cover to the second strap.

The first strap may enclose the rest of the circumference of the right thigh of the user, which the first cover and the first fastening frame do not enclose, and the second strap may enclose the rest of the circumference of the left thigh of the user, which the second cover and the second fastening frame do not enclose. The first and second straps may include, for example, elastic material (e.g., a band).

FIG. 5 is a diagram illustrating components of an electronic system of a wearable device, according to various embodiments.

Referring to FIG. 5, an electronic system of the wearable device 100 may include the control circuit 510, the communication circuit 516, one or more sensors (e.g., the IMU 522, the first angle sensor 524, and the second angle sensor 524-1), driving modules 530 and 530-1, an input circuit 540, the sound output circuit 550, and the haptic circuit 560. At least one of said components (e.g., the input circuit 540, the sound output circuit 550, or the haptic circuit 560) may be omitted from the electronic system, or one or more other components (e.g., a display circuit, a lighting circuit for driving the lighting module 85, or a PMIC) may be added thereto.

The driving module 530 may include a motor 534 and a motor driver circuit 532 for driving the motor 534, and the driving module 530-1 may include a motor 534-1 and a motor driver circuit 532-1 for driving the motor 534-1. Although FIG. 5 illustrates two driving modules, this embodiment is just an example. In specific embodiments, there may be one driving module or three or more driving modules. The driving module 530 including the motor driver circuit 532 and the motor 534 may correspond to the first driving module 45 of FIG. 3, and a driving module 530-1 including the motor driver circuit 532-1 and the motor 534-1 may correspond to the second driving module 35 of FIG. 3.

The one or more sensors may include a sensor configured to acquire sensor data (or sensed values). The one or more sensors may transmit the acquired sensor data to the control circuit 510. The one or more sensors may include, for example, the IMU 522, the first angle sensor 524, and/or the second angle sensor 524-1. Each of the sensors may be provided in plurality, and some of the sensors may be omitted.

The IMU 522 may measure the motion of the body of the user. The IMU 522 may sense the accelerations, angular velocities, and rotation angles (e.g., roll, pitch, and yaw) of x-axis, y-axis, and z-axis according to the motion of the user. The IMU 522 may measure, for example, a pelvic motion of the user. The IMU 522 may measure an anterior-posterior tilt for the forward and backward tilt of the user's pelvis, a lateral oblique for the left and right tilt of the pelvis, and a rotation of the pelvis. The roll, pitch, and yaw measured from the IMU 522 may each correspond to one of the anterior-posterior tilt, the lateral oblique, and the rotation of the pelvis. The pelvic motion of the user may correspond to the motion of a waist support frame (e.g., the waist support frame 20 of FIG. 3) of the wearable device 100. In an embodiment, the IMU 522 may be disposed on a printed circuit board (PCB) in a base body (e.g., the base body 80) of the wearable device 100 and may measure a gradient indicating the degree of tilting of the wearable device 100 and/or the acceleration of the wearable device 100.

In an embodiment, the first angle sensor 524 and the second angle sensor 524-1 may measure hip joint angles according to the leg motion of the user. The first angle sensor 524 may sense the hip joint angle of the right leg of the user, and the second angle sensor 524-1 may sense the hip joint angle of the left leg of the user. Each of the first angle sensor 524 and the second angle sensor 524-1 may include, for example, an encoder and/or a Hall sensor. The hip joint angle of the right leg sensed by the first angle sensor 524 may correspond to the motion (e.g., the angle) of a first torque transmission frame (e.g., the first torque transmission frame 55 of FIG. 3) of the wearable device, and the hip joint angle of the left leg sensed by the second angle sensor 524-1 may correspond to the motion (e.g., the angle) of a second torque transmission frame (e.g., the second torque transmission frame 50 of FIG. 3) of the wearable device.

In an embodiment, the first angle sensor 524 and the second angle sensor 524-1 may be angle sensors configured to sense a knee joint angles or hip joint angles according to the leg motion of the user.

In an embodiment, the processor 512 may determine the angular velocity of the first torque transmission frame by differentiating a change in the angle over time of the first torque transmission frame sensed by the first angle sensor 524, and determine the angular velocity of the second torque transmission frame by differentiating a change in the angle over time of the second torque transmission frame sensed by the second angle sensor 524-1.

In an embodiment, the one or more sensors may further include a torque sensor configured to sense a torque value, a position sensor configured to acquire a position value of the wearable device 100, a proximity sensor configured to detect the proximity of an object, a biosignal sensor configured to detect a biosignal of a user, a distance sensor configured to measure the distance to an object, a pressure sensor configured to measure a pressure value, and/or a temperature sensor configured to measure ambient temperature.

The input circuit 540 may receive instructions or data to be used by a component (e.g., the processor 512) of the wearable device 100 from the outside (e.g., the user) of the wearable device 100. The input circuit 540 may include, for example, a key (e.g., a button) and/or a touch screen.

The sound output circuit 550 may output a sound signal to the outside of the wearable device 100. The sound output circuit 550 may include a speaker that outputs a guide sound signal (e.g., a driving start sound or an operation error notification sound), music content, and/or a guiding voice.

The driving modules 530 and 530-1 may generate external force to be applied to the legs of the user by control of the control circuit 510. The driving modules 530 and 530-1 may be disposed in positions corresponding to the positions of the hip joints of the user and may generate torque to be applied to the legs of the user based on a control signal generated by the control circuit 510. The control circuit 510 may transmit the control signal to the motor driver circuits 532 and 532-1, and the motor driver circuits 532 and 532-1 may control the operation of the motors 534 and 534-1 by generating a current signal (or a voltage signal) corresponding to the control signal and supplying the current signal (or the voltage signal) to the motors 534 and 534-1. The current signal may not be supplied to the motors 534 and 534-1 according to the control signal. The motor driver circuits 532 and 532-1 may convert a direct-current (DC) voltage supplied from a battery into an alternating-current (AC) voltage and may supply the converted voltage to the motors 534 and 534-1. One or more motors (e.g., the motors 534 and 534-1) included in the wearable device 100 may generate torque by control of the processor 512. When the motors 534 and 534-1 are driven as the current signal is supplied to the motors 534 and 534-1, the motors 534 and 534-1 may generate assistance forces for assisting leg motions of the user or resistance forces for hindering leg motions of the user. The motor 534 or 534-1 may generate torque based on electrical energy supplied from the battery. The motor 534 or 534-1 may be, for example, a brushless DC (BLDC) motor or a permanent magnet synchronous motor (PMSM).

The control circuit 510 may control the overall operation of the wearable device 100 and may generate a control signal to control each component of the wearable device 100. The control circuit 510 may include the processor 512 and the memory 514.

The processor 512 may execute software to control at least one other component (e.g., a hardware or software component) of the wearable device directly or indirectly connected to the processor 512 and may perform a variety of data processing or computation. For example, the processor 512 may control the operation of the motors 534 and 534-1. As at least part of data processing or computation, the processor 512 may store instructions or data received from another component (e.g., the communication circuit 516) in the memory 514, process the instructions or data stored in the memory 514, and store result data acquired as the result of processing in the memory 514. The processor 512 may include one or more processors, and the operations of the wearable device 100 described in the present disclosure may be performed by one processor or by a combination of multiple processors.

According to an embodiment, the processor 512 may include a main processor (e.g., a central processing unit (CPU) or an application processor (AP)) or an auxiliary processor (e.g., a graphics processing unit (GPU), a neural processing unit (NPU), an image signal processor (ISP), a sensor hub processor, or a communication processor (CP)) that is operable independently from, or in conjunction with, the main processor. The processor 512 may be implemented as a system on chip (SoC) or an integrated circuit (IC) configured to perform processing. The auxiliary processor may be implemented separately from the main processor or as a part of the main processor.

Herein, each “processor” may include a processing circuit or may include a plurality of processors. For example, as used in the present disclosure including in the claims, the term “processor” may include various processing circuits including at least one processor, in which the at least one processor may be configured to perform the various functions described herein in a distributed manner individually and/or collectively. When the present disclosure describes the “processor”, “at least one processor”, or “one or more processors” as being configured to perform a plurality of functions, these terms include situations where one processor performs some of the functions and the other processors perform the other functions or where a single processor performs all the functions, but embodiments are not limited thereto. In addition, the one or more processors may include a combination of processors that perform various cited/disclosed functions, for example, in a distributed manner. The one or more processors may execute instructions to accomplish or perform various functions.

The memory 514 may store data used by at least one component (e.g., the processor 512) of the wearable device 100. The data may include, for example, software, input data or output data on instructions related thereto, and sensor data. The memory 514 may include at least one instruction executable by the processor 512. The memory 514 may include one or more memories, and the instructions controlled by the processor 512 to perform the operations of the wearable device 100 described herein may be stored in one memory or divided and stored in multiple memories. The memory 514 may include a volatile memory or a non-volatile memory.

The communication circuit 516 may support establishing a direct (e.g., wired) communication channel or a wireless communication channel between the control circuit 510 and another component of the wearable device 100 or an external electronic device (e.g., the electronic device 210 or the other wearable device 220 of FIG. 2) and performing communication via the established communication channel. For example, the communication circuit 516 may transmit the sensor data acquired by the sensor to an external electronic device (e.g., the electronic device 210 of FIG. 2) and receive a control signal from the external electronic device. According to an embodiment, the communication circuit 516 may include one or more CPs that operate independently of the processor 512 and support direct (e.g., wired) communication or wireless communication. According to an embodiment, the communication circuit 516 may include a wireless communication circuit (e.g., a cellular communication circuit, a short-range wireless communication circuit, or a global navigation satellite system (GNSS) communication circuit), and/or a wired communication circuit. The wireless communication circuit may communicate with another component of the wearable device 100 and/or an external device via, for example, Bluetooth™, wireless fidelity (Wi-Fi), infrared data association (IrDA), a legacy cellular network, a 5G network, a next-generation network, the Internet, or a computer network (e.g., a local area network (LAN) or a wide area network (WAN)).

The haptic circuit 560 may provide haptic feedback to the user by control of the processor 512. The haptic circuit 560 may include one or a plurality of haptic actuators. The haptic actuator may include, for example, a piezo actuator, a bander type actuator, and/or a vibration motor-based actuator. One or a plurality of haptic actuators may be provided. In an embodiment, the haptic actuator may be disposed in at least one of the base body, a torque transmission frame (e.g., the first torque transmission frame 55 or the second torque transmission frame 50 of FIG. 3), and a thigh fastening portion (e.g., the first thigh fastening portion 2 or the second thigh fastening portion 1 of FIG. 3) of the wearable device 100.

In an embodiment, the wearable device 100 may operate in a walking ability evaluation mode to evaluate the walking ability of the user. The communication circuit 516 may receive a control signal related to the progress of the walking ability evaluation mode from an electronic device (e.g., the electronic device 210 of FIG. 2) and may transmit the received control signal to the processor 512. The processor 512 may operate the wearable device 100 in the walking ability evaluation mode according to the control signal. The instructions stored in the memory 514 may be executed by the processor 512, and, when the instructions are executed by the processor 512, the processor 512 (or the wearable device 100) may perform the operations of the wearable device 100 described herein. In response to receiving the control signal for performing the walking ability evaluation mode from the electronic device 210 via the communication circuit 516, the processor 512 may perform the walking ability evaluation mode for acquiring sensor data including the motion information of the wearable device 100 without generating torque from one or more motors (e.g., the motor 534 or the motor 534-1).

The processor 512 may control the communication circuit 516 and one or more sensors (e.g., the inertial sensor 522, the first angle sensor 524, or the second angle sensor 524-1) to operate the walking ability evaluation mode. The one or more sensors may acquire the sensor data including the motion information of the wearable device 100 by measuring the motion of the wearable device 100 corresponding to the motion of the user wearing the wearable device 100. The one or more sensors may include the IMU 522 for measuring the motion information on the motion of the wearable device 100 corresponding to the user's pelvic motion and an angle sensor (e.g., the first angle sensor 524 or the second angle sensor 524-1) for measuring the motion information on the motion of the wearable device 100 corresponding to the user's leg motion.

The sensor data acquired by the one or more sensors may be stored in the memory 514. The sensor data may include sensor values according to a time when being output from the IMU 522, the first angle sensor 524, and/or the second angle sensor 524-1. The sensor data may include, for example, a yaw value, a roll value, and a pitch value acquired by the IMU 522. The yaw value, the roll value, and the pitch value may each include information on one of the rotation motion of the pelvis, the anterior-posterior tilt motion of the pelvis, and the lateral oblique motion of the pelvis.

The communication circuit 516 may transmit the sensor data. The processor 512 controls the communication circuit 516 to transmit the sensor data to the electronic device 210 in the user's walking ability evaluation mode such that the electronic device 210 may determine evaluation data on the user's walking posture based on the sensor data and may perform at least one of determining a recommended exercise program and outputting guide content based on the determined evaluation data on the walking posture. The evaluation data on the walking posture may include evaluation data on the user's pelvic motion of the user. The evaluation data on the pelvic motion may include evaluation data on the rotation of the user's pelvis, evaluation data on the anterior-posterior tilt of the pelvis, evaluation data on the lateral oblique of the pelvis, or any combination thereof.

In an embodiment, the processor 512 may determine (or calculate) a gait index based on the sensor data. For example, the processor 512 may determine the gait index including at least one of a gait speed of the user, a stride of the user, a gait cycle of the user, and a gait symmetry index of the user, based on the sensor data. The processor 512 may control the communication circuit 516 to transmit information on the determined gait index to the electronic device 210. According to embodiments, the wearable device 100 may transmit the sensor data to the electronic device 210 without determining the gait index, and the electronic device 210 may determine the gait index as described above. Alternatively, the wearable device 100 may determine only some (e.g., the gait speed or the stride) of the gait index, and the electronic device 210 may determine the rest of the gait index.

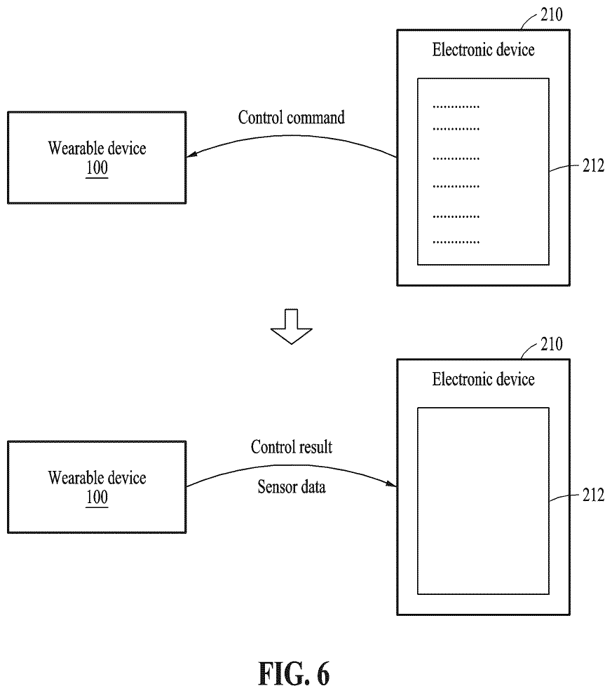

FIG. 6 is a diagram illustrating an interaction between a wearable device and an electronic device, according to various embodiments.

Referring to FIG. 6, the wearable device 100 may communicate with the electronic device 210. For example, the electronic device 210 may be a user terminal (e.g., a smartphone or a tablet personal computer (PC)) of a user who uses the wearable device 100. In an embodiment, the wearable device 100 and the electronic device 210 may be connected to each other through short-range wireless communication (e.g., Bluetooth™ or Wi-Fi communication).

In an embodiment, the electronic device 210 may check a state of the wearable device 100 or may execute an application to control or operate the wearable device 100. A screen of a UI may be displayed to control the operation of the wearable device 100 or determine an operation mode of the wearable device 100 on a display 212 of the electronic device 210 through the execution of the application. The UI may be, for example, a GUI.

In an embodiment, the user may input an instruction for controlling the operation of the wearable device 100 (e.g., an execution instruction to a walking assistance mode or an exercise assistance mode) or change settings of the wearable device 100 through a GUI screen on the display 212 of the electronic device 210. Further, the user may set exercise goals and change a torque parameter to be applied to the wearable device 100 through the GUI screen. The torque parameter may include, for example, a first parameter to control the magnitude of torque generated by a motor (e.g., the motor 534 or the motor 534-1 of FIG. 5) of the wearable device 100 and/or a second parameter to control a point in time when the torque is applied. In various embodiments of the present disclosure, the term “torque parameter” may be replaced with the term “parameter”, “robot parameter”, or “control parameter”. The electronic device 210 may generate a control command (or a control signal) corresponding to an operation control command or setting change command input by the user and may transmit the generated control command to the wearable device 100. In an embodiment, the torque parameter set by the user may be included in the control instruction.

The electronic device 210 may display a UI screen for controlling the operation of the wearable device 100 or measuring the physical ability of the user on the display 212. The user may input a command (e.g., a command to execute a physical ability measurement mode) to control the operation of the wearable device 100 through the UI screen on the display 212 of the electronic device 210. The electronic device 210 may generate a control command corresponding to the command and transmit the generated control command to the wearable device 100. The wearable device 100 may operate according to the received control command and may transmit a control result according to the received control command and/or data (e.g., sensor data or result data processed by the wearable device 100) to the electronic device 210. The electronic device 210 may provide the user with result information (e.g., current exercise state information, exercise result information, exercise posture evaluation information, and physical ability evaluation information) derived by analyzing the data of the wearable device 100 and/or the control result through the display 212. For example, the electronic device 210 may provide the user with walking analysis result content including evaluation information on the walking ability through the GUI screen.

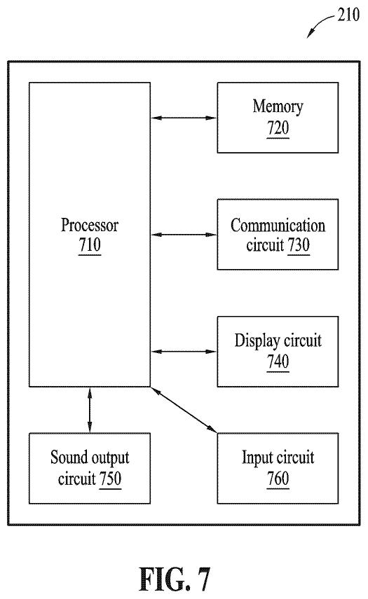

FIG. 7 is a diagram illustrating components of an electronic device, according to various embodiments.

Referring to FIG. 7, the electronic device 210 may include a processor 710, a memory 720, a communication circuit 730, a display circuit 740, a sound output circuit 750, and an input circuit 760. In an embodiment, at least one (e.g., the sound output circuit 750) of these components may be omitted from the electronic device 210, or one or more other components (e.g., a sensor circuit, a haptic circuit, and a battery) may be added thereto.

The processor 710 may control at least one other component (e.g., a hardware or software component) of the electronic device 210, and may perform a variety of data processing or computation. According to an embodiment, as at least part of data processing or computation, the processor 710 may store instructions or data received from another component (e.g., the communication circuit 730) in the memory 720, process the instructions or data stored in the memory 720, and store result data in the memory 720. The processor 710 may include one or more processors, and the operations of the electronic device 210 described in the present disclosure may be performed by one processor or by a combination of multiple processors.

According to an embodiment, the processor 710 may include at least one of a main processor (e.g., a central processing unit (CPU) or an application processor (AP)) or an auxiliary processor (e.g., a graphics processing unit (GPU)), a neural processing unit (NPU), an image signal processor (ISP), a sensor hub processor, or a communication processor (CP)) that is operable independently of, or in conjunction with the main processor. The processor 710 may be implemented as an SoC or an IC configured to perform processing.

The memory 720 may store a variety of data used by at least one component (e.g., the processor 710 or the communication circuit 730) of the electronic device 210. The data may include, for example, a program (e.g., an application) and input data and/or output data on a command related thereto. The memory 720 may include at least one instruction executable by the processor 710. The memory 720 may include one or more memories, and the instructions controlled by the processor 710 to perform the operations of the electronic device 210 described herein may be stored in one memory or divided and stored in multiple memories. The memory 720 may include a volatile memory or a non-volatile memory.

The communication circuit 730 may support establishing a direct (e.g., wired) communication channel or a wireless communication channel between the electronic device 210 and another electronic device (e.g., the wearable device 100, the other wearable device 220, or the server 230) and performing communication via the established communication channel. The communication circuit 730 may include a communication circuit configured to perform a communication function. The communication circuit 730 may include one or more CPs that operate independently of the processor 710 (e.g., an AP) and support direct (e.g., wired) communication or wireless communication. According to an embodiment, the communication circuit 730 may include a wireless communication circuit configured to perform wireless communication (e.g., a Bluetooth communication circuit, a cellular communication circuit, a Wi-Fi communication circuit, or a GNSS communication circuit) or a wired communication circuit (e.g., a LAN communication circuit or a power line communication (PLC) circuit). For example, the communication circuit 730 may transmit a control instruction to the wearable device 100 and receive, from the wearable device 100, at least one of sensor data including body motion information of a user who is wearing the wearable device 100, state data of the wearable device 100, or control result data corresponding to the control instruction.

The display circuit 740 may visually provide information to the outside (e.g., the user) of the electronic device 210. For example, the display circuit 740 may include a display, such as a liquid-crystal display (LCD) or organic light-emitting diode (OLED) display, a hologram device, or a projector device. The display circuit 740 may further include a control circuit configured to control the driving of a display. In an embodiment, the display circuit 740 may further include a touch sensor configured to sense a touch or a pressure sensor configured to measure the magnitude of force generated by the touch. The display circuit 740 may output a UI screen for controlling the wearable device 100 or providing a variety of information (e.g., exercise evaluation information and setting information of the wearable device 100).

The sound output circuit 750 may output sound signals to the outside of the electronic device 210. The sound output circuit 750 may include a guide sound signal (e.g., a driving start sound or an operation error notification sound) based on a state of the wearable device 100 and a speaker for playing musical content or a guiding voice.

The input circuit 760 may receive a command or data to be used by a component (e.g., the processor 710) of the electronic device 210 from the outside (e.g., the user) of the electronic device 210. The input circuit 760 may include an input component circuit and may receive a user input. The input circuit 760 may include, for example, a touch recognition circuit for recognizing a touch on a key (e.g., a button) and/or a screen.

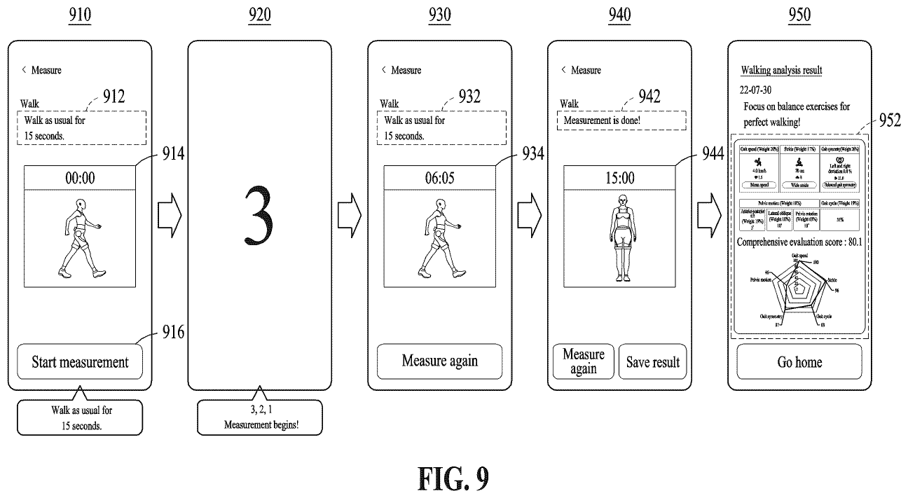

In an embodiment, the electronic device 210 in association with the wearable device 100 may operate a walking ability evaluation mode to evaluate (or measure) the walking ability of the user. The user may command the electronic device 210 to execute the walking ability evaluation mode through the user input. The user may execute an application for performing walking ability evaluation on the electronic device 210 and may set the details for the walking ability evaluation (e.g., a gate time for measurement or a walking ability index to be evaluated) through the application. The processor 710 may provide a UI for guiding the user through an evaluation procedure of the walking ability through a display included in the display circuit 740. The example of the UI is described below with reference to FIG. 9. When receiving a user input to execute the walking ability evaluation mode through the input circuit 760, the processor 710 may control the communication module 730 to transmit a control signal for the execution of the walking ability evaluation mode to the wearable device 100 in response to the received user input. When receiving the control signal for the execution of the walking ability evaluation mode from the electronic device 210, the wearable device 100 may activate the walking ability evaluation mode. The wearable device 100 may acquire sensor data including the motion information of the wearable device 100 corresponding to the body motion of the user in the walking ability evaluation mode. The wearable device 100 may transmit the acquired sensor data to the electronic device 210.

The communication circuit 730 may receive the sensor data measured by one or more sensors of the wearable device 100 from the wearable device 100 worn on the user's body. The sensor data may include motion information on the motion of the wearable device 100 corresponding to the user's body motion. The sensor data may include, for example, a yaw value, a roll value, and a pitch value acquired by an IMU (e.g., the IMU 522 of FIG. 5) of the wearable device 100, in which the yaw value, the roll value, and the pitch value may each include information on any one of the rotation motion of a pelvis, the anterior-posterior tilt motion of the pelvis, and the lateral oblique motion of the pelvis. The sensor data may include an angle value measured by an angle sensor (e.g., the first angle sensor 524 and the second angle sensor 524-1 of FIG. 5) of the wearable device 100. The angle value measured by the angle sensor may include information on the user's leg motion.

The instructions stored in the memory 720 may be executed by the processor 710, and, when the instructions are executed by the processor 710, the processor 710 (or the electronic device 210) may perform the operations of the electronic device 210 described herein. The processor 710 may determine the assessment result of the user's walking ability based on the sensor data received from the wearable device 100. The processor 710 may extract feature points corresponding to reference measurement values for the evaluation of the walking ability from the sensor data and may estimate the walking ability of the user based on the extracted feature points.

In an embodiment, the processor 710 may determine the assessment data of the user's walking posture based on the sensor data received from the wearable device 100. The evaluation data on the walking posture may include evaluation data on the user's pelvic motion during walking. In the present disclosure, the user's “pelvic motion” may correspond to the user's waist motion or upper body motion. The evaluation data on the pelvic motion may include evaluation data on the rotation of the user's pelvis, evaluation data on the anterior-posterior tilt of the pelvis, evaluation data on the lateral oblique of the pelvis, or any combination thereof. In an embodiment, the processor 710 may determine the evaluation data on the pelvic motion according to an equation corresponding to a gait speed range including the user's gait speed among different preset gait speed ranges. Equations for determining evaluation scores for the pelvic motion may be predefined according to gait speeds. Once the user's mean gait speed is determined, evaluation scores for each of the pelvic rotation, the anterior-posterior tilt, and the lateral oblique may be determined according to an equation corresponding to the mean gait speed. A higher evaluation score may indicate a better evaluation result. The evaluation data on the walking posture may further include evaluation data on a gait symmetry determined based on a left step time and a right step time during the user's walking. In an embodiment, the processor 710 may perform at least one of determining a recommended exercise program and outputting guide content based on the determined evaluation data on the walking posture. The guide content may include, for example, audio coaching content provided to the user during the user's exercise.

In an embodiment, the processor 710 may further determine evaluation data on the user's walking activeness. The evaluation data on the walking activeness may include, for example, evaluation data on at least one of the user's gait speed, the user's stride, and the user's gait cycle.

In an embodiment, the processor 710 may extract first feature points corresponding to heel contact and second feature points corresponding to toe off from the sensor data measured by an IMU of the wearable device 100 and may determine the user's gait speed, walking time, stride, and/or gait symmetry index based on the extracted first feature points and second feature points. The processor 710 may determine a step time, a swing time, a stance time, a stride time, and/or a double support time of the user based on a time interval between temporally adjacent first feature points among the first feature points, a time interval between temporally adjacent second feature points among the second feature points, and a time interval between a first feature point and a second feature point that is temporally adjacent to the first feature point.

In an embodiment, the processor 710 may determine a step length, a stride length, and/or a leg length based on sensor data measured by an angle sensor of the wearable device 100. The sensor data measured by the angle sensor may include a hip joint angle of the user, and the processor 710 may determine the step length, the stride length, and/or the leg length based on a hip joint angle value at the time corresponding to heel contact among hip joint angle values (e.g., a hip joint angle value corresponding to a right leg and a hip joint angle value corresponding to a left leg) measured by the angle sensor.

In an embodiment, the processor 710 may determine a sum of step lengths measured in a walking interval and a sum of incurred time and may estimate the user's mean gait speed by dividing the sum of step lengths by the sum of incurred time.

In an embodiment, the processor 710 may calculate a mean value of the left step length and a mean value of the right step length for steps of the user and may determine the gait symmetry index based on a difference between the mean value of the left step length and the mean value of the right step length. The processor 710 may determine the gait symmetry index corresponding to a difference between the step time of the left step and the step time of the right step for the steps of the user.

Although various gait indices are described as being determined by the processor 710 of the electronic device 210, at least some of the gait indices may be determined by a processor (e.g., the processor 512 of FIG. 5) of the wearable device 100. In this case, the wearable device 100 may transmit information on the determined gait index to the electronic device 210 via a communication circuit (e.g., the communication circuit 516 of FIG. 5).

In an embodiment, the processor 710 may determine comprehensive evaluation data for the user's walking based on the evaluation data on the walking posture and the evaluation data on the walking activeness. The evaluation data on the walking activeness may include an evaluation score for the rotation of the user's pelvis, an evaluation score for the anterior-posterior tilt of the pelvis, an evaluation score for the lateral oblique of the pelvis, and an evaluation score for the gait symmetry. The evaluation data on the walking activeness may include an evaluation score for the gait speed, an evaluation score for the stride, and an evaluation score for the gait cycle of the user. In an embodiment, the processor 710 may control the display circuit 740 to output walking analysis result content including the evaluation data on the walking posture and the evaluation data on the walking activeness. The walking analysis result content may include comprehensive evaluation data. The comprehensive evaluation data may include a comprehensive evaluation score determined based on the evaluation data on the walking posture and the evaluation data on the walking activeness. The comprehensive evaluation score may be a score that synthesizes the evaluation results regarding various walking ability indicators of the user. The comprehensive evaluation score may be a score determined based on an evaluation score for the rotation of the pelvis, an evaluation score for the anterior-posterior tilt of the pelvis, an evaluation score for the lateral oblique of the pelvis, an evaluation score for the gait symmetry, an evaluation score for the gait speed, an evaluation score for the stride, and an evaluation score for the gait cycle of the user. A higher comprehensive evaluation score may indicate that the user walks more energetically with better posture.

The processor 710 may perform at least one of determining a recommended exercise program and outputting guide content based on the determined comprehensive evaluation data. In an embodiment, the processor 710 may determine a recommended exercise program by determining a configuration of an exercise mode and an exercise intensity to be applied to the user based on the evaluation data on the walking posture and the evaluation data on the walking activeness. In this case, the exercise mode to be applied to the user may include an assistance mode in which the wearable device 100 generates an assistive force to assist the user's motion while the user is performing an exercise program and a resistance mode in which the wearable device 100 generates a resistive force to impede the user's motion while the user is performing the exercise program. The exercise program may be configured by appropriately combining the assistance mode, the resistance mode, and the exercise intensity depending on an exercise progress section. The size of the assistive force or resistance force output from the wearable device 100 may vary depending on the exercise intensity. In an embodiment, the processor 710 may control the output of a guide voice to guide the user's walking motion according to the evaluation data on the pelvic motion. For example, if the pelvic motion is evaluated to be excessive compared to the user's gait speed, the processor 710 may be controlled to output the guide voice, like “Walk with strength in your abs”, during the user's exercise. If the evaluation of the anterior-posterior tilt of the user's pelvis is not good, the processor 710 may be controlled to output the guide voice, like “Walk with strength in your glutes” during the user's exercise.

As described above, the electronic device 210 may collect sensor data including information on the motion of the user's pelvis during walking through the wearable device 100 and may more precisely evaluate the user's walking posture during their walking based on the information on the pelvic motion. The pelvic motion (or pelvic mobility) is an indicator for evaluating walking efficiency, and a change in the pelvic motion during walking is observed in patients with specific diseases. Accordingly, including the evaluation of the pelvic motion in the evaluation of walking ability enables a more detailed evaluation of the walking ability. Users may receive a more accurate and convenient diagnosis of their walking posture by using the electronic device 210 and the wearable device 100 without the need for an expensive dedicated measurement device (e.g., a medical device) or the assistance of a skilled professional. The electronic device 210 provides a comprehensive indicator of the user's walking ability based on various gait indices measured from the user's walking and may recommend an exercise program customized for the walking evaluation results or may output guide content (e.g., voice coaching content) during exercise to provide a solution for only areas of the user's gait indices that require improvement. Through this process, the electronic device 210 may increase the effectiveness of the user's walking exercise using the wearable device 100 and the user's interest in a walking exercise.

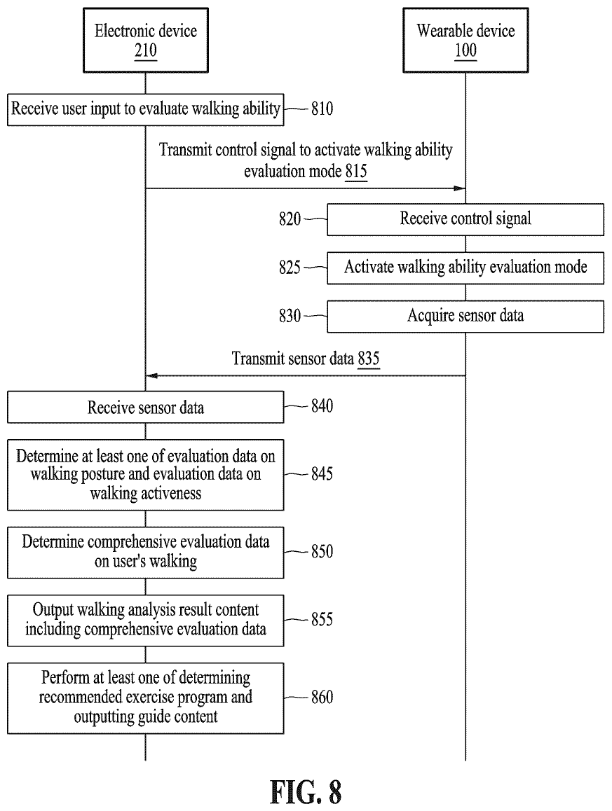

FIG. 8 is a diagram illustrating an operating method of a wearable device and electronic device for assessing the user's walking ability, according to various embodiments. At least one of operations of FIG. 8 may be simultaneously or parallelly performed with one another, and the order of the operations may be changed. In addition, at least one of the operations may be omitted or another operation may be additionally performed.

Referring to FIG. 8, in operation 810, the electronic device 210 may receive a user input to evaluate the walking ability of a user. The user may select a walking ability measurement test to be performed on an application executed on the electronic device 210 and detailed settings (e.g., a measurement time or a gait index to be measured) related to the walking ability measurement test.

In operation 815, the electronic device 210 may transmit a control signal to activate a walking ability evaluation mode to the wearable device 100 in response to receiving the user input in operation 810. In operation 820, the wearable device 100 may receive the control signal to activate the walking ability evaluation mode of the user from the electronic device 210. The control signal may include information on detailed settings related to the walking ability measurement test selected by user input.

In operation 825, the wearable device 100 may activate the walking ability evaluation mode in response to the control signal received in operation 820. In operation 830, the wearable device 100 may acquire sensor data including motion information on the motion of the wearable device 100 corresponding to the user's body motion by using one or more sensors (e.g., the IMU 522, the first angle sensor 524, and the second angle sensor 524-1 of FIG. 5) of the wearable device 100 in the walking ability evaluation mode. In an embodiment, the wearable device 100 may not generate torque through a motor (e.g., the motor 534 and the motor 534-1 of FIG. 5) when operating in the walking ability evaluation mode.

In operation 835, the wearable device 100 may transmit the sensor data to the electronic device 210 via a communication circuit. In operation 840, the electronic device 210 may receive the sensor data from the wearable device 100 in response to the control signal transmitted in operation 815.

In operation 845, the electronic device 210 may determine at least one of evaluation data on a walking posture and evaluation data on walking activeness of the user, based on the received sensor data. The electronic device 210 may extract feature points corresponding to reference measurement values for the measurement of a specific gait index from the sensor data and may estimate the gait index of the user based on the extracted feature points. The evaluation data on the user's walking posture may include evaluation data on a pelvic motion of the user's walking and/or evaluation data on a gait symmetry determined based on a left step time and a right step time during the user's walking. For example, the evaluation data on the walking posture may include an evaluation score for the rotation of the user's pelvis, an evaluation score for the anterior-posterior tilt of the pelvis, an evaluation score for the lateral oblique of the pelvis, and an evaluation score for the gait symmetry. The evaluation data on the walking activeness may include evaluation data on at least one of the user's gait speed, the user's stride, and the user's gait cycle. For example, the evaluation data on the walking activeness may include an evaluation score for the gait speed, an evaluation score for the stride, and an evaluation score for the gait cycle of the user.

In operation 850, the electronic device 210 may determine comprehensive evaluation data on the user's walking based on at least one of the evaluation data on the walking posture and the evaluation data on the walking activeness of the user. The comprehensive evaluation data may include a comprehensive evaluation score determined based on the evaluation data on the walking posture and the evaluation data on the walking activeness.

In operation 855, the electronic device 210 may output walking analysis result content including the comprehensive evaluation data. The user may monitor the walking analysis result content through an application executed on the electronic device 210.

In operation 860, the electronic device 210 may perform at least one of determining a recommended exercise program and outputting guide content based on the determined comprehensive evaluation data. For example, the electronic device 210 may perform at least one of determining a recommended exercise program and outputting guide content based on the determined evaluation data on the walking posture. The electronic device 210 may determine a recommended exercise program by determining a configuration of an exercise mode and an exercise intensity to be applied to the user based on the evaluation data on the walking posture and the evaluation data on the walking activeness and may provide the determined recommended exercise program to the user. In addition, the electronic device 210 may output voice coaching content to guide the user's exercise while the user is wearing the wearable device 100 and exercising. The voice coaching content may provide, for example, a real-time guide for improving the user's walking ability, a notification of a change in an exercise type/exercise intensity, the provision of exercise-related information, and a guide voice related to encouragement. The guide voice may be provided to the user through a sound output circuit (e.g., the sound output circuit 550) of the wearable device 100, a sound output circuit (e.g., the sound output circuit 750 of FIG. 7) of the electronic device 210, and/or another wearable device (e.g., the wireless earphones 222 or the smartwatch 224 of FIG. 2) connected to the electronic device 210.

FIG. 9 is a diagram illustrating UI screens provided to a user through an electronic device when walking ability evaluation is performed, according to various embodiments.

FIG. 9 illustrates the UI screens provided to the user through an application of the electronic device 210 to evaluate the user's walking ability. The user may step on a starting point while wearing the wearable device 100. Until now, a UI screen 910 may be output on the electronic device. The UI screen 910 may include a description 912 of a measurement method for evaluating the walking ability, a time counter 914, and a selection icon 916 for controlling the start of measurement, but the configuration of the UI screen 910 is not limited thereto.

When the user is ready for the measurement, the user may request the start of the measurement by touching the selection icon 916 on the UI screen 910. Then, the electronic device 210 may output a UI screen 920 to countdown a preparation time. While the preparation time is being counted down, the wearable device 100 may initialize (or calibrate) sensor data output from one or more sensors (e.g., the IMU 522, the first angle sensor 524, and the second angle sensor 524-1 of FIG. 5). When the UI screen 910 and the UI screen 920 are provided, the description of the measurement method may be provided through an audio signal.

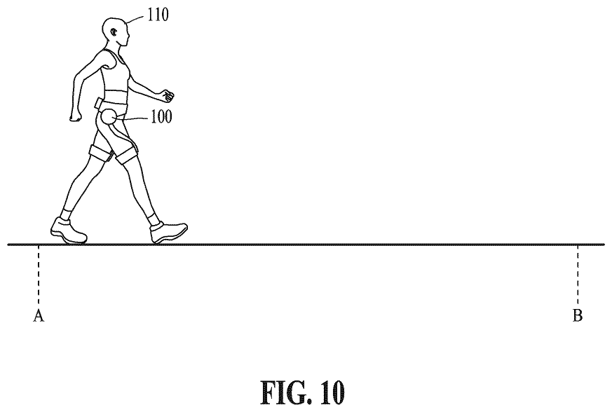

Once the countdown for the preparation time is complete, the user may start walking for a set time (e.g., 15 seconds) while wearing the wearable device 100. The user may walk at a normal gait speed until the measurement time is completed, and, during the user's walking, the wearable device 100 may acquire sensor data according to the user's walking through the one or more sensors. For example, referring to FIG. 10, the user 110 may start walking from a starting point A according to a guide provided by the electronic device 210 and may walk to an end point B where the measurement time is completed. During walking, the wearable device 100 may acquire the sensor data through the one or more sensors and may transmit the acquired sensor data to the electronic device 210. Referring to FIG. 9 again, a UI screen 930 may be provided from the electronic device 210 before the set measurement time is completed after the measurement has started. The UI screen 930 may provide the description 932 of the measurement method and information on an elapsed time 934 since the measurement started up to now.

Once the set measurement time is completed, the collection of the sensor data for evaluating the user's walking ability may be completed. When the measurement for evaluating the walking ability is completed, the electronic device 210 may provide the user with a UI screen 940 including a description 942 to notify the user of the completion of the measurement and information on a set measurement time 944. To notify the completion of the measurement, a notification sound may be output from the electronic device 210 and/or the wearable device 100.

The electronic device 210 may evaluate the user's walking ability by analyzing the sensor data acquired up to the point where the measurement is completed. In an embodiment, the electronic device 210 may receive sensor data from the wearable device 100 in real time while the user is wearing the wearable device 100 and walking and may extract various gait indices based on the received sensor data. The electronic device 210 may determine evaluation data on the walking posture and/or evaluation data on the walking activeness based on the extracted gait indices. When the walking ability evaluation is completed, the electronic device 210 may provide a UI screen 950 including walking analysis result content 952 to the user. The walking analysis result content 952 may include, for example, evaluation data on a pelvic motion, a stride, a gait symmetry, a gait cycle, and a gait speed and a comprehensive evaluation score on the user's walking ability. The gait analysis result content 952 may include a radar chart indicating scores for each of the gait speed, the stride, the gait cycle, the gait symmetry, and the pelvic motion (or pelvic mobility). The UI screen 950 may also include walking-related guide content. For example, the walking-related guide content includes guide comments, like “If you focus on balance exercises, you may achieve a perfect gait!”, “You are walking briskly but with a slightly stiff posture!”, or “Let's try improving the flexibility of the posture by increasing the mobility of the pelvis a little more.”

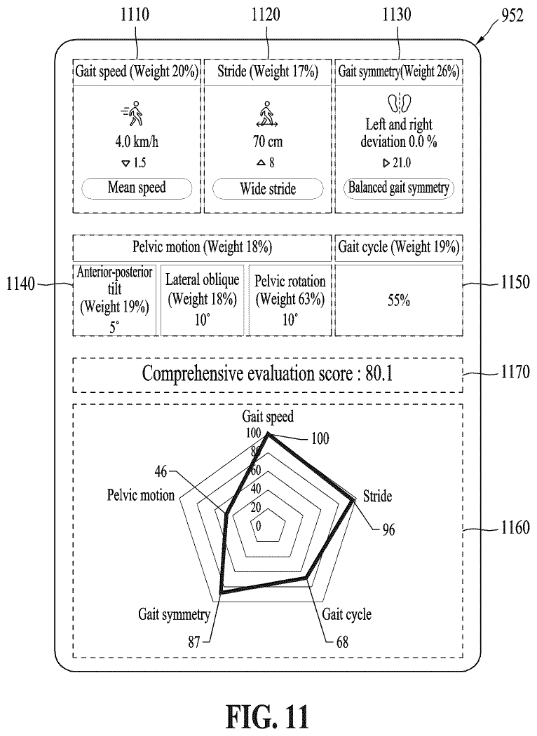

FIG. 11 is a diagram illustrating an example of walking analysis result content according to various embodiments.

Referring to FIG. 11, the walking analysis result content 952 provided to a user may include report information that evaluates the user's walking. For example, the walking analysis result content 952 may include evaluation data 1110 on a gait speed, evaluation data 1120 on a stride, evaluation data 1130 on a gait symmetry, evaluation data 1140 on a pelvic motion during the user's walking, evaluation data 1150 on a gait cycle, and a comprehensive evaluation score 1170, but examples are not limited thereto. Some of them may be omitted from the walking analysis result content 952, and other pieces of evaluation information may be included in the walking analysis result content 952. Evaluation data on each gait index may include an evaluation score determined according to set evaluation criteria, and an evaluation score 1160 of each gait index may be expressed as a radar chart. The radar chart may visually express a relationship between evaluation scores of each of the gait speed, the stride, the gait cycle, the gait symmetry, and the pelvic motion (or pelvic mobility) in polygons.

The evaluation data 1110 on a gait speed may include the evaluation of whether the user's gait speed is appropriate relative to a measured gait speed and a statistical value for a gait speed. The evaluation data 1120 on a stride may include the evaluation of whether the user's stride is appropriate relative to a measured stride and a statistical value for a stride. The evaluation data 1130 on a gait symmetry may include the evaluation of whether the user's gait symmetry is appropriate relative to a measured gait symmetry index (e.g., a time difference between right and left steps) and a statistical value for a gait symmetry. The evaluation data 1140 on a pelvic motion may include a measured pelvic rotation value, a measured pelvic anterior-posterior tilt value, and a measured pelvic lateral oblique value. The evaluation data 1150 on a gait cycle may include a measured gait cycle.

The comprehensive evaluation score 1170 may be determined by applying an evaluation score of each gait index to a predefined equation. In this case, a weight may be applied to each gait index, and the weight applied to each gait index may vary. As the weight applied to a gait index increases, the influence that the gait index has on the comprehensive evaluation score 1170 may increase.

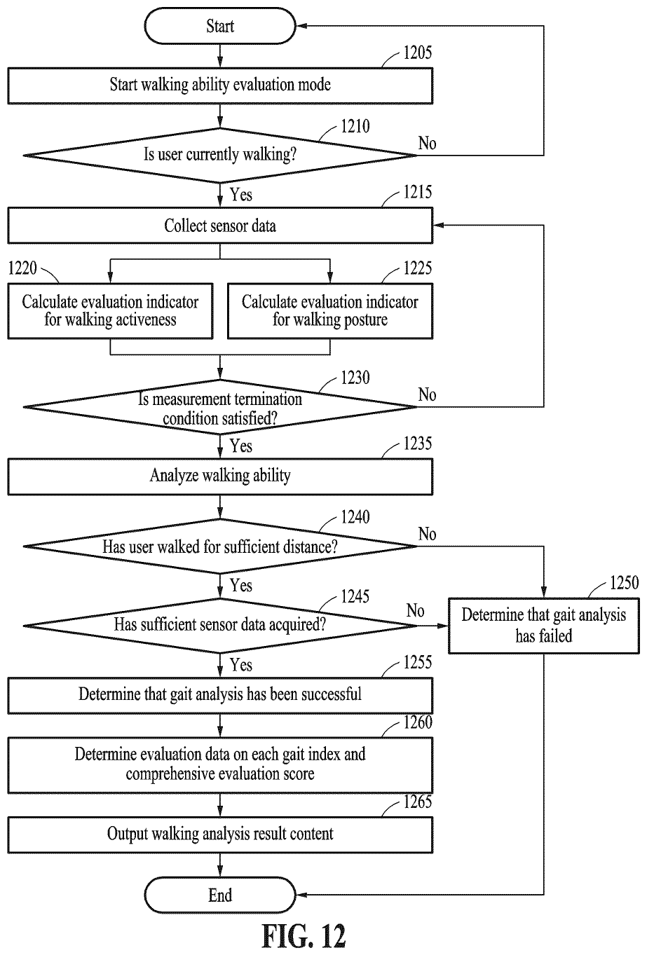

FIG. 12 is a flowchart illustrating operations of a walking ability evaluation process by using a wearable device and electronic device, according to various embodiments. At least one of operations of FIG. 12 may be simultaneously or parallelly performed with one another, and the order of the operations may be changed. In addition, at least one of the operations may be omitted or another operation may be additionally performed.

Referring to FIG. 12, in operation 1205, the electronic device 210 may start a walking ability evaluation mode in response to a user input. The electronic device 210 may transmit a control signal to the wearable device 100 to activate the walking ability evaluation mode.

In operation 1210, the wearable device 100 may determine whether a user is currently walking. For example, the wearable device 100 may measure the number of steps of the user based on sensor data measured from one or more sensors (e.g., the IMU 522, the first angle sensor 524, and the second angle sensor 524-1 of FIG. 5) and may determine the user is walking when the measured number of steps is three or more.

After monitoring for a certain time from the beginning of the walking ability evaluation mode, if the user is determined to be not walking (if “No” in operation 1210), the wearable device 100 and/or the electronic device 210 may provide a user with a notification for re-measurement. If the user is determined to be walking (if “Yes” in operation 1210), the wearable device 100 may collect sensor data through one or more sensors in operation 1215. The wearable device 100 may transmit the collected sensor data to the electronic device 210.

The electronic device 210 may determine one or more gait indices based on the received sensor data. In operation 1220, the electronic device 210 may calculate an evaluation indicator for walking activeness based on the sensor data received from the wearable device 100. For example, the electronic device 210 may calculate an evaluation indicator for each of a gait speed, a stride, and a gait cycle. In operation 1225, the electronic device 210 may calculate an evaluation indicator for a walking posture based on the sensor data received from the wearable device 100. For example, the electronic device 210 may calculate an evaluation indicator for each of a pelvic motion and a gait symmetry.

In an embodiment, the electronic device 210 may identify the moment when the user steps on the ground based on sensor data acquired through an angle sensor of the wearable device 100 and may calculate a step length by using a leg spread angle of the user at the identified moment.

In an embodiment, the electronic device 210 may identify a first point when the user's heel touches the ground and a second point when the user's heel touches the ground again after the foot is off the ground from sensor data received from the wearable device 100 and may determine a time difference between the first point and the second point to be a gait cycle.

In an embodiment, the process of calculating a gait speed is as described below. Sensor data may be extracted from sections (sections determined to be steady walking) of the user's walking section during a measurement time except for an initial acceleration section, a last deceleration section, and sections determined not to be walking motions. The gait speed may be calculated by dividing a walking distance that is calculated based on the extracted sensor data by a time taken to walk that walking distance.

In an embodiment, the calculation process for a gait symmetry index is as described below. The gait symmetry index indicates how symmetrical the user's left and right leg steps are during walking. Based on the sensor data of the sections determined to be steady walking of the user's walking section during the measurement time, a left step time which is the time the user walks with the left leg and a right step time which is the time the user walks with the right leg may be measured. The gait symmetry index may be calculated based on the time difference between the left step time and the right step time. As the time difference is smaller, the gait symmetry index may be determined to be a higher value. The gait symmetry index may be calculated through, for example, Equation 1 as shown below.

$$100 \times \frac{\vert Left Step Time Mean - Right Step Time Mean \vert}{0.5 \cdot ( Left Step Time Mean + Right Step Time Mean )} \qquad [ Equation 1 ]$$

Here, LeftStepTimeMean represents the mean of the left step times measured per left step, and RightStepTimeMean represents the mean of the right step times measured per right step.

A high gait symmetry index refers to a high gait symmetry or a good gait symmetry.

In an embodiment, the gait speed, gait cycle, and gait symmetry indices are calculated by a processor (e.g., the processor 512 of FIG. 5) of the wearable device 100, and information on the calculated gait speed, gait cycle, and gait symmetry indices may be transmitted to the electronic device 210. The wearable device 100 may determine the user's gait speed, gait cycle, and gait symmetry indices by using a machine-learning model having the sensor data of an IMU and an angle sensor as inputs and the gait speed, gait cycle, and gait symmetry indices as outputs. The machine-learning model may be a neural network trained to output the gait speed, gait cycle, and gait symmetry indices based on inputs.

In operation 1230, the electronic device 210 may determine whether a measurement termination condition is satisfied. For example, the electronic device 210 may determine that the measurement termination condition is satisfied when a set measurement time (e.g., 15 seconds) has elapsed after the walking ability evaluation mode begins.

If the measurement termination condition is satisfied (if “Yes” in operation 1230), the electronic device 210 may analyze the user's walking ability in operation 1235. The electronic device 210 may perform the walking ability analysis based on the evaluation indicator for walking activeness calculated in operation 1220 and the evaluation indicator for a walking posture calculated in operation 1225.

In operation 1240, the electronic device 210 may determine whether the user has walked for a sufficient distance (e.g., 10 m) in the measurement process. If it is determined that the user has walked the sufficient distance (if “Yes” in operation 1240), the electronic device 210 may determine whether the wearable device 100 has acquired a sufficient amount of sensor data to perform gait analysis in operation 1245. In an embodiment, operations 1240 and 1245 may also be performed by the wearable device 100. If it is determined that the sufficient amount of sensor data has been acquired (if “Yes” in operation 1245), the electronic device 210 may determine that the gait analysis in the gait ability assessment mode has been successful in action 1255.

If it is determined that the user has not walked the sufficient distance (if “No” in operation 1240), or it is determined that the wearable device 100 has not acquired the sufficient amount of sensor data (if “No” in operation 1245), the electronic device 210 may determine that the gait analysis in the gait ability assessment mode has failed in operation 1250.

If the gait analysis is determined to be successful, the electronic device 210 may determine evaluation data on each gait index and a comprehensive evaluation score in operation 1260. The electronic device 210 may determine an evaluation score for each gait index and may determine the comprehensive evaluation score based on the result of applying a weight to the evaluation score of each gait index. For example, a weighted sum of the evaluation scores of gait indices may be determined to be the comprehensive evaluation score. The weights applied to the evaluation scores of the gait indices may be derived by an analytic hierarchy process (AHP). The comprehensive evaluation score may also be adjusted by a correction value determined by a mean and a standard deviation of each gait index.

In an embodiment, a gait speed, a stride, and a gait cycle may be determined to have higher evaluation scores as the calculated values thereof are greater. A pelvic motion and a gait symmetry may be determined to have higher evaluation scores as the calculated values are closer to a specific target value. The evaluation score of each gait index may be determined by using a probability distribution by the characteristics of each gait index. For example, the evaluation scores of gait indices of a gait speed, a stride, and a gait cycle may be assigned with 70 points if a measured value falls within the mean section of statistical distributions of respective gait indices, 100 points if it falls within the top 15% or higher section, and 0 points if it falls within the bottom 0.5% or lower section. For example, the evaluation scores of gait indices of a pelvic motion and a gait symmetry may be assigned with 70 points if a difference between a measured value and a set target value falls within the mean section of statistical distributions of respective gait indices, 100 points if it falls within the upper 15% or higher section, and 0 points if it falls within the lower 0.5% or lower section.

In operation 1265, the electronic device 210 may output walking analysis result content (e.g., the walking analysis result content 952 of FIG. 11) including evaluation data on each gait index and the comprehensive evaluation score.

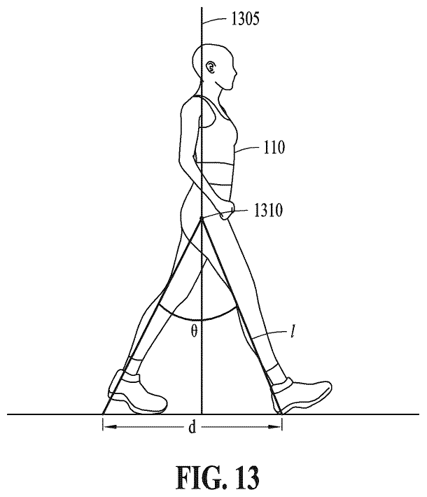

FIG. 13 is a diagram illustrating measuring a user's stride and gait speed based on sensor data, according to various embodiments.

Referring to FIG. 13, the electronic device 210 may estimate gait indices, such as a step length, a stride length, and a leg length of the user 110 based on sensor data acquired from an angle sensor of the wearable device 100. The sensor data acquired from the angle sensor may include information on the user's hip joint angle value.

The length of a right leg and a left leg from a position 1310 of a hip joint of the user 110 is L, and a sum of an angle formed with a line 1305 perpendicular to the ground passing through the position 1310 of the hip joint when the left leg of the user 110 touches the ground and an angle formed with the line 1305 when the right leg of the user 110 touches the ground is defined as a step angle θ. The electronic device 210 may estimate the user's step length d based on Equation 2 as shown below.

$$d = 2 \times l \times sin \frac{θ}{2} \qquad [ Equation 2 ]$$

If a step angle acquired for each gait cycle while the user walks during a measurement time is θ₁, θ₂, . . . , θₙ, the user may estimate the user's leg length l based on the Equation 3 as shown below. Once the user's leg length l is estimated, gait parameters (e.g., a stride) may be calculated based on the leg length l.

$$10 = 2 \cdot l \cdot ( sin ( \frac{{θ}_{1}}{2} ) + sin ( \frac{{θ}_{2}}{2} ) + … + sin ( \frac{{θ}_{n}}{2} ) ) \qquad [ Equation 3 ]$$

$$l = \frac{5}{{\sum }_{k = 1}^{n} sin ( \frac{{θ}_{k}}{2} )}$$

In an embodiment, the electronic device 210 may extract a steady walking section from the measured whole walking section of the user. In an embodiment, the electronic device 210 may extract a walking section based on the remaining sensor data, excluding sensor data acquired near the beginning and end of the walking. The electronic device 210 may estimate a mean gait speed of the user 110 by determining a sum of the estimated one step strides and a sum of incurred time in the extracted walking section and dividing the sum of one step strides by the sum of incurred time.

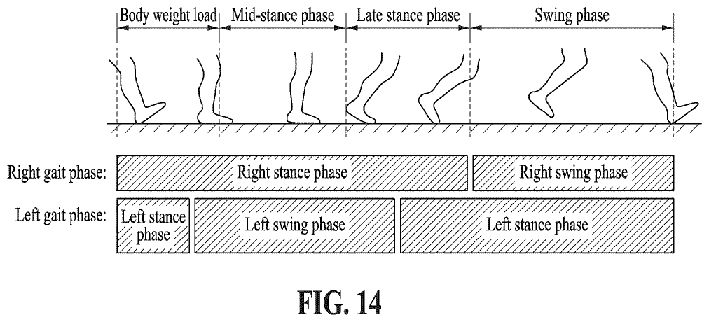

FIG. 14 is a diagram illustrating measuring a user's gait cycle based on sensor data, according to various embodiments.

Referring to FIG. 14, gait phases of either leg of the user for the user's walking may be predefined. The gait phases may include, for example, a stance phase and a swing phase. The gait phases of the left leg may be divided into a left stance phase (LSt) and a left swing phase (LSw). The gait phases of the right leg may be divided into a right stance phase (RSt) and a right swing phase (RSw). The term “gait phase” may be used interchangeably with a “gait state”.

The stance phase and the swing phase may be further subdivided into a plurality of phases. For example, the stance phase may be subdivided into initial contact, a weight bearing, a middle stance phase, a terminal stance phase, and a pre-swing phase. The swing phase may be subdivided into an initial swing phase, a middle swing phase, and a terminal swing phase. The stance phase and the swing phase may be subdivided differently depending on embodiments and are not limited to the embodiments described herein.

According to a general gait mechanism, the stance phase and the swing phase are performed alternately during the user's gait. In the transition of a normal gait phase, based on the occurrence sequence of events indicating the beginning of each gait phase, the transition of gait phases are in the order of a RSt, a LSw, a LSt, and a RSw. After the RSw, the RSt is performed again.

In an embodiment, the electronic device 210 may identify the time (the time when the RSt begins) when the user's right heel touches the ground to the time (the time when the RSw ends and the RSt begins again) when the right foot leaves and the right heel touches the ground again from the sensor data received from the wearable device 100 and may determine a time difference between the identified times as the gait cycle. Alternatively, the electronic device 210 may identify the time (the time when the LSt begins) from when the user's left heel touches the ground to the time (the time when the LSw ends and the LSt begins again) when the left foot leaves and the left heel touches the ground again and may determine a time difference between the identified times as the gait cycle.

In an embodiment, the electronic device 210 may determine the gait cycle based on a time ratio occupied by the swing phase in a time interval including the swing phase and the stance phase of either foot. As the time ratio occupied by the swing phase is smaller, the gait speed is slower, and the gait symmetry is lower. If the time ratio occupied by the swing phase is less than defined normal criteria, an evaluation score for the gait cycle may be assigned low, and, if the time ratio is greater than the defined normal criteria, the evaluation score for the gait cycle may be assigned high.

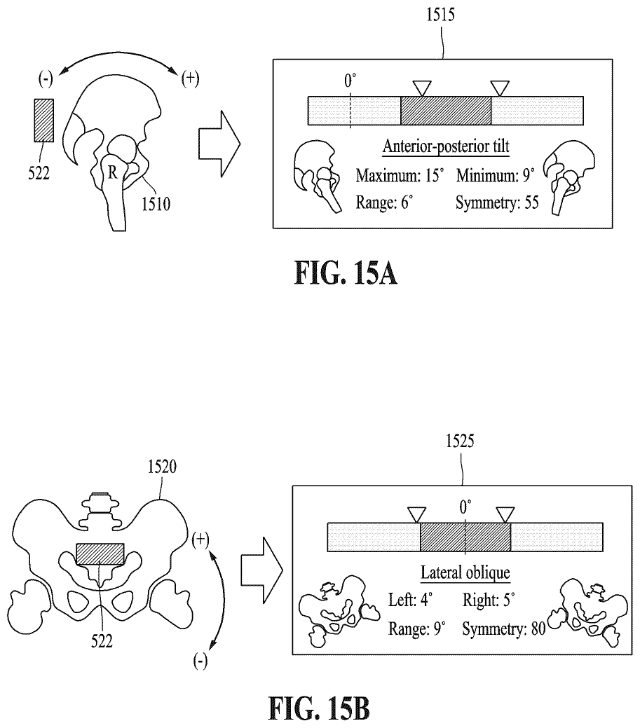

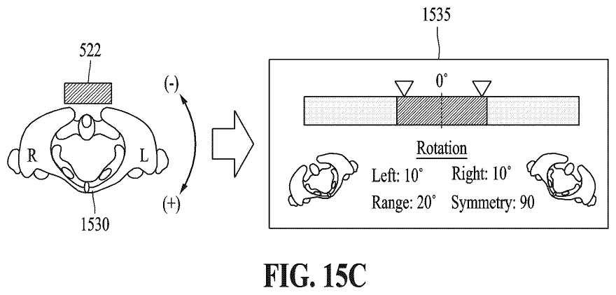

FIGS. 15A, 15B, and 15C are diagrams each illustrating determining evaluation data on a user's pelvis motion based on sensor data, according to various embodiments.

Referring to FIG. 15A, the electronic device 210 may determine evaluation data on an anterior-posterior tilt of a pelvis 1510 of the user during walking based on the sensor data of the IMU 522 received from the wearable device 100. In an embodiment, the IMU 522 is positioned within a base body (e.g., the base body 80 of FIG. 3) of the wearable device 100 and may measure a pelvic motion behind the user's pelvis. The IMU 522 may measure a yaw value, a roll value, and a pitch value according to the user's pelvic motion, and any one of these values may indicate information on the anterior-posterior tilt of the pelvis 1510. For example, a change in the pitch value may indicate a change in the anterior-posterior tilt of the pelvis 1510. The electronic device 210 may analyze the sensor data of the IMU 522 and may provide the user with analysis result content 1515 including the evaluation data on the anterior-posterior tilt of the pelvis 1510. The analysis results content 1515 may include information on a maximum or high value of the measured anterior-posterior tilt, a minimum or low value of the measured anterior-posterior tilt, a range between the minimum or low value and the maximum or high value, and a symmetry index for the pelvic motion. The symmetry index for the pelvic motion may be a motion similarity that indicates a correlation between a left pelvic motion and a right pelvic motion. The analysis result content 1515 may include visual content as a bar showing a comparison between the maximum value and minimum value of the measured anterior-posterior tilt and a statistical mean value of the anterior-posterior tilt.

Referring to FIG. 15B, the electronic device 210 may determine evaluation data on a lateral oblique of a pelvis 1520 of the user during walking based on the sensor data of the IMU 522 received from the wearable device 100. Any one of a yaw value, a roll value, and a pitch value measured by the IMU 522 may indicate information on the lateral oblique of the pelvis 1520. For example, a change in the roll value may indicate a change in the lateral oblique of the pelvis 1520. The electronic device 210 may analyze the sensor data of the IMU 522 and may provide the user with analysis result content 1515 including the evaluation data on the lateral oblique of the pelvis 1510. Analysis results content 1525 may include information on a left maximum value of the measured lateral oblique, a right maximum value of the measured lateral oblique, a range between the left maximum value and the right maximum value, and a symmetry index for a pelvic motion. The analysis result content 1525 may include visual content as a bar showing a comparison between the right maximum value and the left maximum value of the measured lateral oblique and a statistical mean value of the lateral oblique.

Referring to FIG. 15C, the electronic device 210 may determine evaluation data on a rotation of a pelvis 1530 of the user during walking based on the sensor data of the IMU 522 received from the wearable device 100. Any one of a yaw value, a roll value, and a pitch value measured by the IMU 522 may indicate information on the rotation of the pelvis 1530. For example, a change in the yaw value may indicate a change in the rotation of the pelvis 1530. The electronic device 210 may analyze the sensor data of the IMU 522 and may provide the user with analysis result content 1535 including the evaluation data on the rotation of the pelvis 150. The analysis results content 1535 may include information on a measured left maximum rotation value, a measured right maximum rotation value, a range between the left maximum rotation value and the right maximum rotation value, and a symmetry index of a pelvic motion. The analysis result content 1535 may include visual content as a bar showing a comparison between the measured right maximum rotation value and the left maximum rotation value and a statistical mean value of a pelvic rotation angle during walking.

The gait indices (the anterior-posterior tilt, the lateral oblique, and the rotation) for the pelvic motion determined based on the sensor data may include errors. For example, errors may occur in the sensor data measured by the IMU 522 due to the user's physical characteristics, an interaction between the wearable device 100 and the user's body, an exercise intensity, a gait speed, and/or a worn state of the wearable device 100. The electronic device 210 may perform an error correction process on the sensor data measured by the IMU 522 to reduce the impact of these errors. The electronic device 210 may perform an error correction process for the gait indices for the pelvic motion based on Equation 4 as shown below.

$${Range}_{corrected} = α \times {Range}_{EX 1} + β \qquad [ Equation 4 ]$$

Here, Range_{EX1} denotes an initial gait index for the pelvic motion determined based on the sensor data, and Range_{corrected} denotes a gait index for the pelvic motion with error correction being performed. αdenotes a coefficient applied to Range_{EX1}, β is a constant. For example, may be a value corresponding to a weighted sum of the user's mean gait speed, a maximum angle difference between the right and left legs during walking, an exercise intensity, a stride, the user's body mass index (BMI), and the initial gait index for the pelvic motion before error correction is performed.

The electronic device 210 may determine evaluation data on the pelvic motion based on gait indices for the pelvic motion on which error correction has been performed. The electronic device 210 may precisely evaluate the user's walking posture by evaluating the user's walking ability based on the gait indices for the pelvic motion and may enable the distinguishing of walking problems of patients with musculoskeletal posture problems.

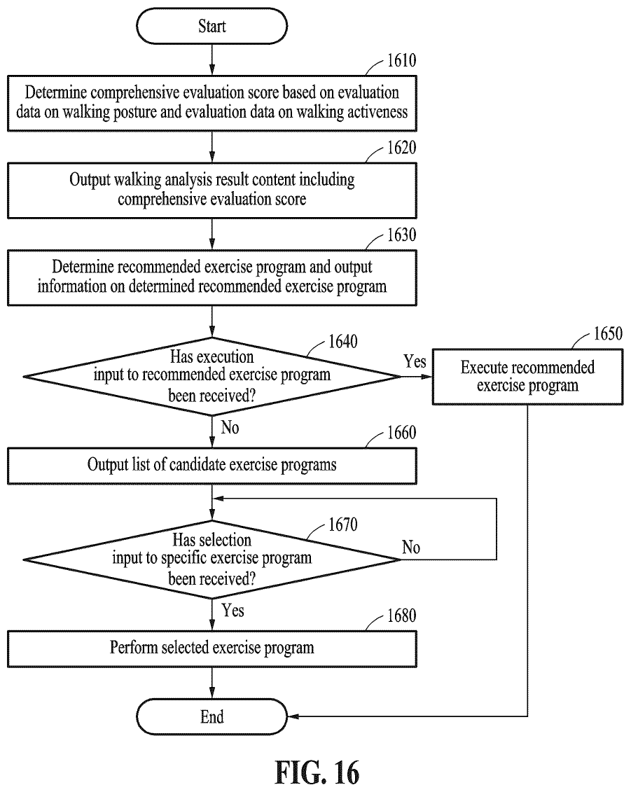

FIG. 16 is a flowchart illustrating operations of a method of providing a recommended exercise program based on a walking ability evaluation result, according to various embodiments. At least one of operations of FIG. 16 may be simultaneously or parallelly performed with one another, and the order of the operations may be changed. In addition, at least one of the operations may be omitted or another operation may be additionally performed.

Referring to FIG. 16, in operation 1610, when the measurement of walking ability is completed, the electronic device 210 may determine comprehensive evaluation data based on evaluation data on a walking posture and evaluation data on walking activeness of a user. The evaluation data on the user's walking posture may include evaluation data on a pelvic motion of the user's walking and/or evaluation data on a gait symmetry determined based on a left step time and a right step time during the user's walking. The evaluation data on the walking activeness may include evaluation data on at least one of the user's gait speed, the user's stride, and the user's gait cycle. The electronic device 210 may determine an evaluation score for each gait index and may determine the comprehensive evaluation score based on the result of applying a weight to the evaluation score of each gait index.

In operation 1620, the electronic device 210 may output walking analysis result content including a comprehensive evaluation score. The user may monitor the walking analysis result content through an application executed on the electronic device 210.

In operation 1630, the electronic device 210 may determine a recommended exercise program to be recommended to the user based on evaluation data on each gait index and may output information on the determined recommended exercise program through the application. The recommended exercise program may be determined by determining a configuration of an exercise mode and an exercise intensity to be applied to the user based on the evaluation data on the walking posture and the evaluation data on the walking activeness among exercise programs that the user may perform while wearing the wearable device 100. The application may provide the user with information on the name, description, and composition of the recommended exercise program.

In operation 1640, the electronic device 210 may determine whether an execution input to the recommended exercise program has been received through a user input.

If it is determined that the execution input to the recommended exercise program has been received (if “Yes” in operation 1640), the electronic device 210 may be controlled to execute the recommended exercise program in operation 1650. The electronic device 210 may control the wearable device 100 to perform the recommended exercise program. The wearable device 100 may control torque that is output from a motor (e.g., the motor 534 and the motor 534-1 of FIG. 5) according to the recommended exercise program. The electronic device 210 and/or the wearable device 100 may also output voice coaching content during the performance of the recommended exercise program.

If it is determined that the execution input to the recommended exercise program has not been received (if “NO” in operation 1640), the electronic device 210 may output a list of candidate exercise programs including a plurality of candidate exercise programs in operation 1660.

In operation 1670, the electronic device 210 may determine whether a selection input to a specific exercise program from the list of candidate exercise programs has been received through the user input. If it is determined that the execution input to the specific exercise program has been received (if “Yes” in operation 1670), the electronic device 210 may be controlled to perform the selected exercise program in operation 1680. The wearable device 100 may control the torque that is output from the motor according to the selected exercise program.

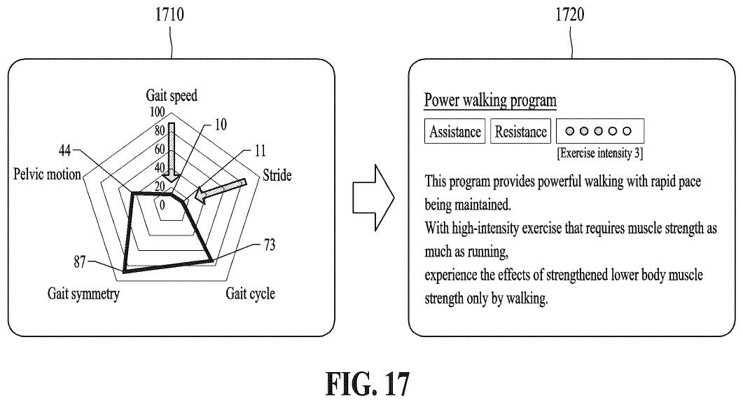

FIG. 17 is a diagram illustrating a recommended exercise program provided based on a walking ability evaluation result, according to various embodiments.

Referring to FIG. 17, the electronic device 210 may determine a recommended exercise program 1720 based on an evaluation score 1710 of each gait index included in a user's walking ability evaluation result determined through a walking ability evaluation. The electronic device 210 may determine a recommended exercise program based on areas where improvement is required in terms of a walking posture and/or walking activeness. The electronic device 210 may determine the recommended exercise program 1720 by determining a configuration and an exercise intensity of an exercise mode (e.g., an assistance mode or a resistance mode) to be applied to the user according to the evaluation score 1710 of each gait index and may provide the determined recommended exercise program 1720 to the user. For example, if evaluation scores for the user's gait speed and stride are low in the evaluation score 1710 of each gait index, the electronic device 210 may determine a “power walking program” as the recommended exercise program 1720 to improve the user's gait speed and stride and may suggest the user to perform the determined power walking program. If it is determined that the user's walking needs to be improved to an active walking posture based on the evaluation score 1710 of each gait index, the electronic device 210 may determine the recommended exercise program centered on the assistance mode. If it is determined that a flexible walking posture needs to be generated by strengthening the user's muscles in the user's walking according to the walking ability evaluation result, the electronic device 210 may determine the recommended exercise program centered on the resistance mode. The electronic device 210 may automatically select the recommended exercise program according to the evaluation score of each gait index. If the evaluation score for the walking activeness is low, the electronic device 210 may determine the recommended exercise program centered on the assistance mode with the objective of increasing flexibility and mobility. In this case, as the evaluation score for the walking activeness is lower, the exercise intensity in the assistance mode may be set higher. With the assistance mode being set high, the wearable device 100 may help increase flexibility and mobility during the user's exercise.

If the evaluation score for the walking posture is low, the electronic device 210 may determine the recommended exercise program centered on the resistance mode with the objective of strengthening muscle strength. In this case, as the evaluation score for the walking posture is lower, the exercise intensity in the resistance mode may be set weaker. With the resistance mode being set weak, the wearable device 100 may help the user gradually strengthen their muscles.

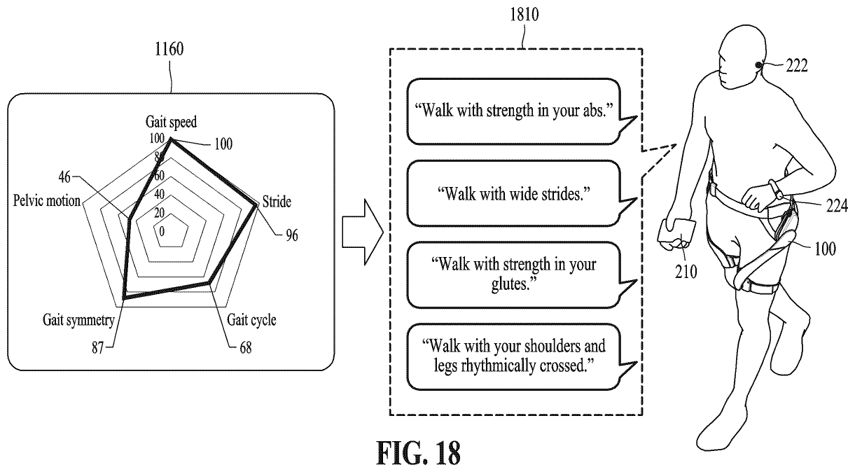

FIG. 18 is a diagram illustrating voice coaching content provided based on a walking ability evaluation result, according to various embodiments.

Referring to FIG. 18, the electronic device 210 may output guide content during the user's exercise based on an evaluation score 1160 of each gait index included in the user's walking ability evaluation result determined through a walking ability evaluation. For example, an exercise assistance system including the wearable device 100, the electronic device 210, and another wearable device (e.g., the wireless earphones 222 or the smartwatch 224) may perform a voice coaching function by providing voice coaching content for the user's exercise (e.g., a walking exercise). The voice coaching function may be implemented through a voice-based human-robot interaction (HRI) and may provide a real-time guidance for improving the user's walking ability, a guidance for changing an exercise type/intensity, the provision of exercise-related information, and a guidance voice related to encouragement.

In an embodiment, when the wireless earphones 222 and/or the smartwatch 224 are worn on the user's body and connected to the electronic device 210 and/or the wearable device 100, the voice coaching content for the voice coaching function may be output through the wireless earphones 222 and/or the smartwatch 224. If the wireless earphones 222 and the smartwatch 224 are not connected, the voice coaching content may be output through the electronic device 210 and/or the wearable device 100. The output control of the voice coaching content may be performed by the electronic device 210.

In an embodiment, the electronic device 210 may enhance the user's exercise effectiveness by providing voice coaching content 1810 to the user while the user is performing an exercise program. The voice coaching content 1810 may be provided to the user periodically. If an evaluation score for a pelvic motion is low in the evaluation score 1160 of each gait index, the electronic device 210 may output the voice coaching content 1810 to correct the user's pelvic motion correctly while the user is performing the exercise program. The output voice coaching content may vary depending on evaluation factors of the pelvic motion resulting in low evaluation scores. For example, if the pelvic motion is evaluated as being excessive compared to a gait speed, the voice coaching content, like “Walk with strength in your abs” may be output. If an evaluation score for a pelvic rotation is low, the voice coaching content, like “Walk with wide strides” may be output. If an evaluation score for an anterior-posterior tilt of the pelvis is low, the voice coaching content, like “Walk with strength in your glutes” may be output. If an evaluation score for a lateral oblique of the pelvis is low, the voice coaching content, like “Walk with your shoulders and legs rhythmically crossed” may be output. Such voice coaching content may be output during the performance of a recommended exercise program that has been recommended to the user by the electronic device 210 based on the walking ability evaluation result. The voice coaching content output along with the performance of recommended exercise program may further enhance exercise effects and induce the user's active participation in the exercise.

According to an embodiment, the electronic device 210 may include the communication circuit 730 configured to receive sensor data measured by one or more sensors of the wearable device 100 worn on the user's body, the memory 720 configured to store instructions, and one or more processors 710.

The instructions, when executed by the one or more processors 710, may cause the one or more processors 710 to determine evaluation data on the user's walking posture based on the received sensor data and perform at least one operation of determining a recommended exercise program and outputting guide content based on the determined evaluation data on the user's walking posture.

The sensor data may include motion information on the wearable device 100's motion corresponding to the user's body motion.

The evaluation data on the user's walking posture may include evaluation data on a pelvis motion of the user's walking.

The evaluation data on the pelvis motion may include one or a combination of evaluation data on rotation of the user's pelvis, evaluation data on an anterior-posterior tilt of the pelvis, and evaluation data on a lateral oblique of the pelvis.

The instructions, when executed by the one or more processors 710, may cause the one or more processors 710 to determine evaluation data on the user's walking activeness, determine comprehensive evaluation data on the user's walking based on the evaluation data on the walking posture and the evaluation data on the walking activeness, and perform at least one operation of determining a recommended exercise program and outputting guide content based on the comprehensive evaluation data.

The evaluation data on the walking activeness may include evaluation data on at least one of the user's gait speed, the user's stride, and the user's gait cycle.

The evaluation data on the walking posture may further include evaluation data on a gait symmetry determined based on a left step time and a right step time of the user's walking.

The evaluation data on the walking posture may further include an evaluation score on the rotation of the pelvis, an evaluation score on the anterior-posterior tilt of the pelvis, an evaluation score on the lateral oblique of the pelvis, and an evaluation score on the gait symmetry.

The evaluation data on the walking activeness may further include an evaluation score on the gait speed, an evaluation score on the stride, and an evaluation data on the user's gait cycle.

The instructions, when executed by the one or more processors 710, may cause the one or more processors 710 to determine the recommended exercise program by determining a composition of an exercise mode and an exercise intensity to be applied to the user according to the evaluation data on the walking posture and the evaluation data on the walking activeness.

The exercise mode may include an assistance mode in which the wearable device 100 is configured to generate an assistive force to assist the user's motion while the user performs an exercise program and a resistance mode in which the wearable device 100 is configured to generate a resistive force to impede the user's motion while the user is performing an exercise program.

The electronic device 210 may further include a display circuit 740 including a display.

The instructions, when executed by the one or more processors 710, may cause the one or more processors 710 to control the display circuit 740 to output walking analysis result content including the evaluation data on the walking posture and the evaluation data on the walking activeness.

The walking analysis result content may include the comprehensive evaluation data determined based on the evaluation data on the walking posture and the evaluation data on the walking activeness.

The sensor data may include a yaw value, a roll value, and a pitch value acquired by an IMU of the wearable device 100.

Each of the yaw value, the roll value, and the pitch value may include information on any one of a rotation motion of the pelvis, an anterior-posterior tilt motion of pelvis, and a lateral oblique motion of the pelvis.

The instructions, when executed by the one or more processors 710, may cause the one or more processors 710 to determine the evaluation data on the pelvis motion according to an equation corresponding to a gait speed range including the user's gait speed among preset different gait speed ranges.

The instructions, when executed by the one or more processors 710, may cause the one or more processors 710 to control the wearable device 100 to output a guide voice to guide the user's walking operation according to the evaluation data on the pelvis motion.

According to an embodiment, the wearable device 100 may include one or more sensors configured to acquire sensor data including motion information on the wearable device 100 by measuring a motion of the wearable device 100 corresponding to a motion of a user wearing the wearable device 100, the communication circuit 516 configured to transmit the sensor data, the memory 514 configured to store instructions, and one or more processors 512.

The instructions, when executed by the one or more processors 512, may cause the one or more processors 512 to, in the user's walking ability evaluation mode, control the communication circuit 516 to transmit the sensor data to an electronic device 210 and enable the electronic device 210 to determine evaluation data on the user's walking posture based on the sensor data and perform at least one operation of determining a recommended exercise program and outputting guide content based on the determined evaluation data on the walking posture.

The instructions, when executed by the one or more processors 512, may cause the one or more processors 512 to determine a gait index including at least one of the user's gait speed, the user's stride, the user's gait cycle, and the user's gait symmetry index based on the sensor data and control the communication circuit 516 to transmit information on the determined gait index to the electronic device 210.

The one or more sensors may include the IMU 522 configured to measure motion information on the wearable device 100's motion corresponding to the user's pelvis motion and the angle sensor 524 or 524-1 configured to measure motion information on the wearable device 100's motion corresponding to the user's leg motion.

The wearable device 100 may further include one or more motors configured to generate torque by control of the one or more processors 512.

The instructions, when executed by the one or more processors 512, may cause the one or more processors 512 to, in response to receiving a control signal to perform a walking ability evaluation mode from the electronic device 210 via the communication circuit 516, perform a walking ability evaluation mode configured to acquire the sensor data including the motion information of the wearable device 100 without generating torque from the one or more motors.

According to an embodiment, an operating method of an electronic device may include operation 810 of receiving a user input for a user's walking ability evaluation, operation 815 of transmitting a control signal to activate a walking ability evaluation mode to a wearable device in response to the receiving of the user input, operation 840 of receiving sensor data including motion information on the wearable device's motion corresponding to the user's body motion from the wearable device in response to the transmitting of the control signal, operation 850 of determining evaluation data on the user's walking posture based on the received sensor data, and operation 860 of performing at least one operation of determining a recommended exercise program and outputting guide content based on the determined evaluation data on the user's walking posture.

The operating method of the electronic device 210 may further include determining evaluation data on the user's walking activeness and determining comprehensive evaluation data on the user's walking based on the evaluation data on the walking posture and the evaluation data on the walking activeness.

The performing of the at least one operation of determining a recommended exercise program and outputting guide content may include performing at least one operation of determining a recommended exercise program and outputting guide content based on the comprehensive evaluation data.

The performing of the at least one operation of determining a recommended exercise program and outputting guide content may include determining the recommended exercise program by determining a composition of an exercise mode and an exercise intensity to be applied to the user according to the evaluation data on the walking posture and the evaluation data on the walking activeness.

According to an embodiment, a non-transitory computer-readable storage medium may store a program configured to perform the operating method of the electronic device 210.

It should be appreciated that various embodiments of the present disclosure and the terms used therein are not intended to limit the technological features set forth herein to particular embodiments and include various changes, equivalents, or replacements for a corresponding embodiment. In connection with the description of the drawings, like reference numerals may be used for similar or related components. It is to be understood that a singular form of a noun corresponding to an item may include one or more of the things, unless the relevant context clearly indicates otherwise. As used herein, “A or B”, “at least one of A and B”, “at least one of A or B”, “A, B or C”, “at least one of A, B and C”, and “A, B, or C,” each of which may include any one of the items listed together in the corresponding one of the phrases, or all possible combinations thereof. Terms such as “first”, “second”, or “first” or “second” may simply be used to distinguish the component from other components in question, and do not limit the components in other aspects (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., by wire), wirelessly, or via at least a third element(s).

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to various example embodiments, the module may be implemented in the form of an application-specific integrated circuit (ASIC). Thus, each “module” herein may comprise circuitry.

Each embodiment herein may be used in combination with any other embodiment(s) described herein.

Various embodiment as set forth herein may be implemented as software (e.g., a program) including one or more instructions stored in a storage medium that is readable by a machine (e.g., the wearable device 100 of FIG. 1, the electronic device 210 of FIGS. 2 and 7, or the server 230 of FIG. 2). For example, a processor (e.g., the processor 512 of FIG. 5 or the processor 710 of FIG. 7) of the machine may invoke at least one of the one or more instructions stored in the storage medium and execute it. This allows the machine to be operated to perform at least one function according to the at least one instruction invoked. The one or more instructions may include code generated by a compiler or code executable by an interpreter. The machine-readable storage medium may be provided in the form of a non-transitory storage medium. Here, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

The software may include a computer program, a piece of code, an instruction, or some combination thereof, to independently or uniformly instruct or configure the processing device to operate as desired. Software and data may be embodied permanently or temporarily in any type of machine, component, physical or virtual equipment, or computer storage medium or device capable of providing instructions or data to or being interpreted by the processing device. The software also may be distributed over network-coupled computer systems so that the software is stored and executed in a distributed fashion. The software and data may be stored by one or more non-transitory computer-readable recording mediums.

According to an example embodiment, a method according to embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various example embodiments, each component (e.g., a module or a program) of the above-described components may include a single entity or multiple entities, and some of the multiple entities may be separately disposed in different components. According to an embodiment, one or more of the above-described components may be omitted, or one or more other components may be added. Alternatively or additionally, a plurality of components (e.g., modules or programs) may be integrated into a single component. In such a case, according to an embodiment, the integrated component may still perform one or more functions of each of the components in the same or similar manner as they are performed by a corresponding one among the components before the integration. According to various embodiments, operations performed by the module, the program, or another component may be carried out sequentially, in parallel, repeatedly, or heuristically, or one or more of the operations may be executed in a different order or omitted, or one or more other operations may be added.

The units described herein may be implemented using a hardware component, a software component and/or a combination thereof. A processing device may be implemented using one or more general-purpose or special-purpose computers, such as, for example, a processor, a controller and an arithmetic logic unit (ALU), a digital signal processor (DSP), a microcomputer, a field-programmable gate array (FPGA), a programmable logic unit (PLU), a microprocessor, or any other device capable of responding to and executing instructions in a defined manner. The processing device may run an operating system (OS) and one or more software applications that run on the OS. The processing unit also may access, store, manipulate, process, and generate data in response to execution of the software. For purpose of simplicity, the description of a processing unit is used as singular; however, one skilled in the art will appreciate that a processing unit may include multiple processing elements and multiple types of processing elements. For example, the processing unit may include a plurality of processors, or a single processor and a single controller. In addition, different processing configurations are possible, such as parallel processors.

The above-described hardware devices may be configured to act as one or more software modules in order to perform the operations of the above-described examples, or vice versa.

Although the present disclosure exemplifies and describes with reference to various embodiments, it shall be construed that various embodiments are for the illustrative purpose rather than limiting. It shall be further understood by those skilled in the art that various changes in forms and details may be made without departing from the true spirit and full scope of this disclosure including the scope of the attached claims and their equivalents. In addition, it shall be construed that the embodiments described herein may be used with other embodiments of the present disclosure.

1. An electronic device comprising:
a communication circuit configured to receive sensor measured by one or more sensors of a wearable device;
a memory configured to store instructions; and
one or more processors comprising processing circuitry,
wherein the instructions, when executed by the one or more processors individually and/or collectively, cause the one or more processors to:
determine evaluation data on a walking posture of the user based on the received sensor data, and
perform at least one operation determining a recommended exercise program and outputting guide content based on the determined evaluation data on the walking posture of the user,
wherein the sensor data comprises motion information on motion of the wearable device corresponding to a body motion of the user,
wherein the evaluation data on the walking posture of the user comprises evaluation data on a pelvis motion of a pelvis of the user, and
wherein the evaluation data on the pelvis motion comprises at least one of: evaluation data on rotation of the pelvis, evaluation data on an anterior-posterior tilt of the pelvis, or evaluation data on a lateral oblique of the pelvis.

2. The electronic device of claim 1, wherein
the instructions, when executed by the one or more processors individually and/or collectively, cause the one or more processors to:
determine evaluation data on walking activeness of the user, determine comprehensive evaluation data on walking of the user based on the evaluation data on the walking posture and the evaluation data on the walking activeness, and
perform at least one operation of determining a recommended exercise program and outputting guide content based on the comprehensive evaluation data.

3. The electronic device of claim 2, wherein
the evaluation data on the walking activeness comprises evaluation data on at least one of a gait speed of the user, a stride of the user, or a gait cycle of the user.

4. The electronic device of claim 3, wherein
the evaluation data on the walking posture further comprises evaluation data on a gait symmetry based on a left step time and a right step time of the walking of the user.

5. The electronic device of claim 4, wherein
the evaluation data on the walking posture further comprises an evaluation score on the rotation of the pelvis, an evaluation score on the anterior-posterior tilt of the pelvis, an evaluation score on the lateral oblique of the pelvis, and an evaluation score on the gait symmetry, and the evaluation data on the walking activeness further comprises an evaluation score on the gait speed, an evaluation score on the stride, and an evaluation data on the gait cycle of the user.

6. The electronic device of claim 2, wherein
the instructions, when executed by the one or more processors individually and/or collectively, cause the one or more processors to determine the recommended exercise program at least by determining a composition of an exercise mode and an exercise intensity to be applied to the user according to the evaluation data on the walking posture and the evaluation data on the walking activeness,
wherein the exercise mode comprises an assistance mode in which the wearable device is configured to generate an assistive force to assist a motion of the user while the user performs an exercise program and a resistance mode in which the wearable device is configured to generate a resistive force to impede the motion of the user while the user is performing an exercise program.

7. The electronic device of claim 2, further comprising:
a display circuit comprising a display, wherein
the instructions, when executed by the one or more processors individually and/or collectively, cause the one or more processors to control the display circuit to output walking analysis result content comprising the evaluation data on the walking posture and the evaluation data on the walking activeness, and
wherein the walking analysis result content comprises the comprehensive evaluation data determined based on the evaluation data on the walking posture and the evaluation data on the walking activeness.

8. The electronic device of claim 1, wherein
the sensor data comprises a yaw value, a roll value, and a pitch value acquired by an inertial measurement unit (IMU) sensor of the wearable device, and
wherein each of the yaw value, the roll value, and the pitch value comprises information on any one of a rotation motion of the pelvis, an anterior-posterior tilt motion of pelvis, and a lateral oblique motion of the pelvis.

9. The electronic device of claim 1, wherein
the instructions, when executed by the one or more processors individually and/or collectively, cause the one or more processors to determine the evaluation data on the pelvis motion according to an equation corresponding to a gait speed range comprising the gait speed of the user among preset different gait speed ranges.

10. The electronic device of claim 1, wherein
the instructions, when executed by the one or more processors individually and/or collectively, cause the one or more processors to control the wearable device to output a guide voice to guide a walking operation of the user according to the evaluation data on the pelvis motion.

11. A wearable device comprising:
one or more sensors configured to acquire sensor data comprising motion information on the wearable device at least by measuring a motion of the wearable device corresponding to a motion of a user;
a communication circuit configured to transmit the sensor data;
a memory configured to store instructions; and
one or more processors comprising processing circuitry,
wherein the instructions, when executed by the one or more processors individually and/or collectively, cause the one or more processors to:
in a walking ability evaluation mode, control the communication circuit to transmit the sensor data to an electronic device to enable the electronic device to determine evaluation data on a walking posture of the user based on the sensor data and perform at least one operation determining a recommended exercise program and outputting guide content based on the determined evaluation data on the walking posture,
wherein the evaluation data on the walking posture of the user comprises evaluation data on a pelvis motion of a pelvis of the user, and
wherein the evaluation data on the pelvis motion comprises at least one of: evaluation data on rotation of the user's pelvis, evaluation data on an anterior-posterior tilt of the pelvis, or evaluation data on a lateral oblique of the pelvis.

12. The wearable device of claim 11, wherein
the instructions, when executed by the one or more processors individually and/or collectively, cause the one or more processors to determine a gait index comprising at least one of a gait speed of the user, a stride of the user, a gait cycle of the user, or a gait symmetry index of the user based on the sensor data, and
control the communication circuit to transmit information on the determined gait index to the electronic device.

13. The wearable device of claim 11, wherein
the one or more sensors comprise:
an inertial measurement unit (IMU) sensor configured to measure motion information on the motion of the wearable device corresponding to the pelvis motion of the user; and
an angle sensor configured to measure motion information on the motion of the wearable device corresponding to a leg motion of the user.

14. The wearable device of claim 11, further comprising:
one or more motors configured to generate torque by control of the one or more processors, wherein
the instructions, when executed by the one or more processors individually and/or collectively, cause the one or more processors to, in response to receiving a control signal to perform the walking ability evaluation mode from the electronic device via the communication circuit, perform the walking ability evaluation mode configured to acquire the sensor data comprising the motion information of the wearable device without generating torque from the one or more motors.

15. An operating method of an electronic device, the operating method comprising:
receiving a user input for a walking ability evaluation;
transmitting a control signal to activate the walking ability evaluation mode to a wearable device in response to the receiving of the user input;
receiving sensor data comprising motion information on a motion of the wearable device corresponding to a body motion of the user from the wearable device in response to the transmitting of the control signal;
determining evaluation data on a walking posture of the user based on the received sensor data; and
determining a recommended exercise program and outputting guide content based on the determined evaluation data on the walking posture, wherein the evaluation data on the walking posture comprises evaluation data on a pelvis motion of a pelvis of the user, and
the evaluation data on the pelvis motion comprises at least one of evaluation data on rotation of the pelvis of the user, evaluation data on an anterior-posterior tilt of the pelvis, or evaluation data on a lateral oblique of the pelvis.

16. The operating method of claim 15, further comprising:
determining evaluation data on walking activeness of the user; and
determining comprehensive evaluation data on a walking of the user based on the evaluation data on the walking posture and the evaluation data on the walking activeness,
wherein the performing of the determining a recommended exercise program and outputting guide content comprises
performing at least one operation of determining a recommended exercise program and outputting guide content based on the comprehensive evaluation data.

17. The operating method of claim 16, wherein
the evaluation data on the walking activeness comprises evaluation data on at least one of a gait speed of the user, a stride of the user, or a gait cycle of the user, and
the evaluation data on the walking posture comprises evaluation data on a gait symmetry determined based on a left step time and a right step time of the walking of the user.

18. The operating method of claim 17, wherein
the evaluation data on the walking posture further comprises an evaluation score on the rotation of the pelvis, an evaluation score on the anterior-posterior tilt of the pelvis, an evaluation score on the lateral oblique of the pelvis, and an evaluation score on the gait symmetry, and the evaluation data on the walking activeness further comprises an evaluation score on the gait speed, an evaluation score on the stride, and an evaluation data on the gait cycle of the user.

19. The operating method of claim 16, wherein
the performing of the determining a recommended exercise program and outputting guide content comprises determining the recommended exercise program at least by determining a composition of an exercise mode and an exercise intensity to be applied to the user according to the evaluation data on the walking posture and the evaluation data on the walking activeness, and
wherein the exercise mode comprises an assistance mode in which the wearable device is configured to generate an assistive force to assist the motion of the user while the user performs an exercise program and a resistance mode in which the wearable device is configured to generate a resistive force to impede the motion of the user while the user is performing an exercise program.

20. A non-transitory computer-readable storage medium storing instructions that, when executed by one or more processors individually and/or collectively, cause the one or more processors to perform the operating method of claim 15.
